(19) United States
(12) Patent Application Publication
(10) Pub. No.: US 2007/0142304 A1
(43) Pub. Date: Jun. 21, 2007

(54) Diphenylazetidinone derivatives possessing chloesterol absorption inhibitory activity

(76) Inventors: Susanne Alenfalk, Molndal (SE); Mikael Dahlstrom, Molndal (SE); Fana Hunegnaw, Molndal (SE); Staffan Karlsson, Molndal (SE); Malin Lemurell, Molndal (SE); Ann-Margret Lindqvist, Molndal (SE); Tore Skjaret, Molndal (SE); Ingemar Starke, Molndal (SE)

Correspondence Address: Pepper Hamilton LLP, 500 Grant Street, One Mellon Bank Center, 50th Floor, Pittsburgh, PA, 15219-2502, US

(21) Appl. No.: 10/596,725

(22) PCT Filed: Dec. 21, 2004
(86) PCT No.: PCT/SE04/01959

§ 371 Date: Oct. 26, 2006

(30) Foreign Application Priority Data
Dec. 23, 2003 (GB) 0329778.5

(51) Int. Cl. A61K 38/04; C07K 5/08

(52) U.S. Cl. 514/19; 540/200

(57) ABSTRACT

Compounds of formula (XV): [Chemical formula should be inserted here. Please see paper copy] (XV) (wherein variable groups are as defined within) pharmaceutically acceptable salts, solvates, solvates of such salts and prodrugs thereof and their use as cholesterol absorption inhibitors for the treatment of hyperlipidaemia are described. Processes for their manufacture and pharmaceutical compositions containing them are also described.

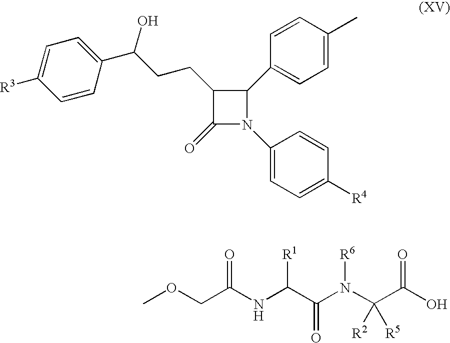

This invention relates to 2-azetidinone derivatives, or pharmaceutically acceptable salts, solvates, solvates of such salts and prodrugs thereof. These 2-azetidinones possess cholesterol absorption inhibitory activity and are accordingly of value in the treatment of disease states associated with hyperlipidaemic conditions. They are therefore useful in methods of treatment of a warm-blooded animal, such as man. The invention also relates to processes for the manufacture of said 2-azetidinone derivatives, to pharmaceutical compositions containing them and to their use in the manufacture of medicaments to inhibit cholesterol absorption in a warm-blooded animal, such as man. A further aspect of this invention relates to the use of the compounds of the invention in the treatment of dyslipidemic conditions.

Atherosclerotic coronary artery disease is a major cause of death and morbidity in the western world as well as a significant drain on healthcare resources. It is well-known that hyperlipidaemic conditions associated with elevated concentrations of total cholesterol and low density lipoprotein (LDL) cholesterol are major risk factors for cardiovascular atherosclerotic disease (for instance “Coronary Heart Disease: Reducing the Risk; a Worldwide View” Assman G., Carmena R. Cullen P. et al; Circulation 1999, 100, 1930-1938 and “Diabetes and Cardiovascular Disease: A Statement for Healthcare Professionals from the American Heart Association” Grundy S, Benjamin I., Burke G., et al; Circulation, 1999, 100, 1134-46).

The concentration of plasma cholesterol depends on the integrated balance of endogenous and exogenous pathways of cholesterol metabolism. In the endogenous pathway, cholesterol is synthesized by the liver and extra hepatic tissues and enters the circulation as lipoproteins or is secreted into bile. In the exogenous pathway cholesterol from dietary and biliary sources is absorbed in the intestine and enters the circulation as component of chylomicrons. Alteration of either pathway will affect the plasma concentration of cholesterol.

The precise mechanism by which cholesterol is absorbed from the intestine is however not clear. The original hypothesis has been that cholesterol is crossing the intestine by unspecific diffusion. But more recent studies are suggesting that there are specific transporters involved in the intestinal cholesterol absorption. (See for instance New molecular targets for cholesterol-lowering therapy Izzat, N. N., Deshazer, M. E. and Loose-Mitchell D. S. JPET 293:315-320, 2000.)

A clear association between reduction of total cholesterol and (LDL) cholesterol and decreased instance of coronary artery disease has been established, and several classes of pharmaceutical agents are used to control serum cholesterol. There major options to regulate plasma cholesterol include (i) blocking the synthesis of cholesterol by agents such as IMG-CoA reductase inhibitors, for example statins such as simvastatin and fluvastatin, which also by up-regulation of LDL-receptors will promote the cholesterol removal from the plasma; (ii) blocking the bile acid reabsorption by specific agents resulting in increased bile acid excretion and synthesis of bile acids from cholesterol with agents such as bile acid binders, such as resins e.g. cholestyramine and cholestipol; and (iii) by blocking the intestinal uptake of cholesterol by selective cholesterol absorption inhibitors. High density lipoprotein (HDL) elevating agents such as fibrates and nicotinic acid analogues have also been employed.

Even with the current diverse range of therapeutic agents, a significant proportion of the hypercholesterolaemic population is unable to reach target cholesterol levels, or drug interactions or drug safety preclude the long term use needed to reach the target levels. Therefore there is still a need to develop additional agents that are more efficacious and are better tolerated.

Compounds possessing such cholesterol absorption inhibitory activity have been described, see for instance the compounds described in WO 93/02048, WO 94/17038, WO 95/08532, WO 95/26334, WO 95/35277, WO 96/16037, WO 96/19450, WO 97/16455, WO 02/50027, WO 02/50060, WO 02/50068, WO 02/50090, WO 02/66464, US 5756470, U.S. Pat. No. 5,767,115 and U.S. RE37721.

The present invention is based on the discovery that certain 2-azetidinone derivatives surprisingly inhibit cholesterol absorption. Such properties are expected to be of value in the treatment of disease states associated with hyperlipidaemic conditions. The compounds of the present invention are not disclosed in any of the above applications and we have surprisingly found that the compounds of the present invention possess beneficial efficacious, metabolic and toxicological profiles that make them particularly suitable for in vivo administration to a warm blooded animal, such as man. In particular certain compounds of the present invention have a low degree of absorption compared to compounds of the prior art whilst retaining their ability to inhibit cholesterol absorption.

Accordingly there is provided a compound of formula (I): 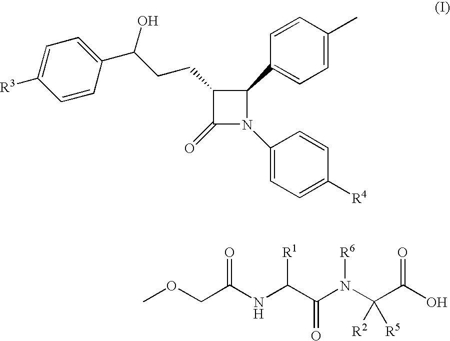
wherein:

R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆alkyl)₂amino, C₁-C₆ alkylcarbonylamino C₁₋₆alkylS(O)ₐ wherein a is 0-2, C₃₋₆ cycloalkyl or aryl; and wherein any aryl group may be optionally substituted by one or two substituents selected from halo, hydroxy, C₁₋₆alkyl or C₁₋₆alkoxy;

R² and R⁵ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy,(C₁-C₄)₃Si, N—(C₁₋₆alkyl)amino, N,N—(C₁₋₆alkyl)₂amino, C₁₋₆alkylS(O)ₐ, C₃₋₆cycloalkyl, aryl or aryl C₁₋₆alkylS(O)ₐ, wherein a is 0-2; and wherein any aryl group may be optionally substituted by one or two substituents selected from halo, hydroxy, C₁₋₆alkyl or C₁₋₆alkoxy;

R³ is hydrogen, alkyl, halo, C₁₋₆alkoxy or C₁₋₆ alkylS—;

R⁴ is hydrogen, C₁₋₆ alkyl, halo or C₁₋₆alkoxy;

R⁶ is hydrogen, C₁₋₆ alkyl, or arylC₁₋₆ alkyl;

wherein R⁵ and R² may form a ring with 2-7 carbon atoms and wherein R⁶ and R² may form a ring with 3-6 carbon atoms;

or a pharmaceutically acceptable salt, solvate, solvate of such a salt or a prodrug thereof.

According to an aspect of the invention R¹ may be hydrogen, phenyl or a branched or unbranched C₁₋₆alkyl. According an aspect of the invention R² may be hydrogen, a branched or unbranched C₁₋₆alkyl, C₃₋₆cycloalkyl or aryl; wherein said C₁₋₆alkyl may be optionally substituted by one or more hydroxy, amino, acylamino, C₁₋₆alkoxyl, halo, methoxy or C₁₋₆alkylS(O)ₐ wherein a is 0-2, C₃₋₆cycloalkyl or aryl; and wherein any aryl group may be optionally substituted by hydroxy, alkyl, alkoxy or cyano. According to an aspect of the invention R³ may be R³ is hydrogen, methyl, chlorine, fluorine, C₁₋₆ alkylS—, or methoxy. According to an aspect of the R⁴ is hydrogen or halo, for instance chlorine or fluorine. According to an aspect of the invention R⁶ is hydrogen, C₁₋₆ alkyl, arylC₁₋₆alkyl,or R⁶ and R² form a ring with 3-6 carbon atoms.

According to a further aspect of the invention:

R¹ is hydrogen;

R² is a branched or unbranched C₁₋₄alkyl, optionally substituted by a C₃₋₆cycloalkyl, alkylS—, aryl optionally substituted by hydroxy or cyano, amino, N—(C₁₋₆alkyl)amino, N,N—(C₁₋₆-alkyl)₂amino or aryl C₁₋₆ alkylS(O)ₐ, wherein a is 0-2

R³ and R⁴ are halo;

R⁵ and R⁶ are hydrogen.

In this specification the term “alkyl” includes both straight and branched chain alkyl groups but references to individual alkyl groups such as “propyl” are specific for the straight chain version only. For example, “C1-6alkyl” and “C₁₋₄alkyl” include propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals, for example “phenylC₁₋₆alkyl” would include benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

The term “aryl” refers to a 4-10 membered aromatic mono or bicyclic ring containing 0 to 5 heteroatoms independently selected from nitrogen, oxygen or sulphur. The term “aryl includes both unsubstituted and substituted aromatic rings. Examples of aryls include phenyl, pyrrolyl, furanyl, imidazolyl, triazolyl, tetrazolyl, pyrazinyl, pyrimidinyl, pyridazinyl, pyridyl, isoxazolyl, oxazolyl, 1,2,4 oxadiazolyl, isothiazolyl, thiazolyl, 1,2,4-triazolyl, thienyl, naphthyl, benzofuranyl, benzimidazolyl, benzthienyl, benzthiazolyl, benzisothiazolyl, benzoxazolyl, benzisoxazolyl, 1,3-benzodioxolyl, indolyl, pyridoimidazolyl, pyrimidoimidazolyl, quinolyl, isoquinolyl, quinoxalinyl, quinazolinyl, phthalazinyl, cinnolinyl and naphthyridinyl. Particularly “aryl” refers to phenyl, thienyl, pyridyl, imidazolyl or indolyl.

Examples of “C₁₋₆alkoxy” include methoxy, ethoxy and propoxy. Examples of “C₁₋₆alkylS(O)ₐ wherein a is 0 to 2” include methylthio, ethylthio, methylsulphinyl, ethylsulphinyl, mesyl and ethylsulphonyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. “C₃₋₆cycloalkyl” refers to cyclopropyl, cyclobutyl, cyclopentyl and cyclohexyl.

A suitable pharmaceutically acceptable salt of a compound of the invention, or other compounds disclosed herein, is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric, acetate or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The invention also provides for a compound of formula (XV): 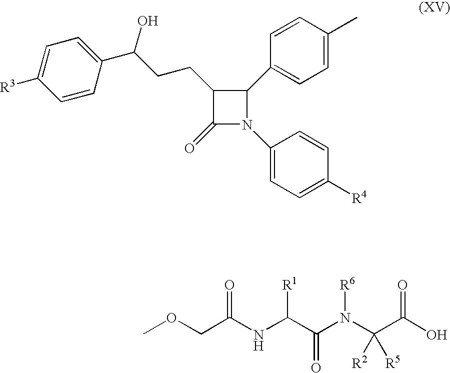
The same substituents apply for the compound of formula (XV) as for those described in connection with the compound of formula (I). The same definitions and other description of formula (I) will also apply to formula (XV). A process for preparing a compound of formula (XV) will be obvious to those skilled in the art, from the description of the process for preparing a compound of formula (I).

The compounds of the formula (I), or other compounds disclosed herein, may be administered in the form of a pro-drug which is broken down in the human or animal body to give a compound of the formula (I). examples of pro-drugs include in vivo hydrolysable esters and in vivo hydrolysable amides of a compound of the formula (I).

An in vivo hydrolysable ester of a compound of the formula (I), or other compounds disclosed herein, containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I), or other compounds disclosed herein, containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

A suitable value for an in vivo hydrolysable amide of a compound of the formula (I), or other compounds disclosed herein, containing a carboxy group is, for example, a N—C₁₋₆alkyl or N,N-di-C₁₋₆alkyl amide such as N-methyl, N-ethyl, N-propyl, N,N-dimethyl, N-ethyl-N-methyl or N,N-diethyl amide.

Some compounds of the formula (I) may have chiral centres and/or geometric isomeric centres (E- and Z-isomers), and it is to be understood that the invention encompasses all such optical, diastereoisomers and geometric isomers that possess cholesterol absorption inhibitory activity.

The invention relates to any and all tautomeric forms of the compounds of the formula (I) that possess cholesterol absorption inhibitory activity.

It is also to be understood that certain compounds of the formula (I) can exist in solvated as well as unsolvated forms such as, for example, hydrated forms. It is to be understood that the invention encompasses all such solvated forms which possess cholesterol absorption inhibitory activity.

Particular values are as follows. Such values may be used where appropriate with any of the definitions, claims or embodiments defined hereinbefore or hereinafter.

R¹ is selected from hydrogen, C₁₋₆alkyl or aryl wherein said C₁₋₆alkyl may be optionally substituted by aryl.

R¹ is selected from C₁₋₆alkyl wherein said C₁₋₆alkyl may be optionally substituted by aryl.

R¹ is selected from hydrogen, isobutyl, phenyl or benzyl.

R¹ is selected from isobutyl or benzyl.

R¹ is hydrogen.

R¹ is isobutyl.

R¹ is phenyl.

R¹ is benzyl.

R² is selected from C₁₋₆alkyl, C₃₋₆cycloalkyl or aryl; wherein said C₁₋₆alkyl may be optionally substituted by one or more hydroxy, amino, guanidino, carboxy, carbamoyl, C₁₋₆alkylS(O)ₐ wherein a is 0, C₃₋₆cycloalkyl or aryl; and wherein any aryl group may be optionally substituted by one substituent selected from hydroxy.

R² is selected from C₁₋₆alkyl or aryl; wherein said C₁₋₆alkyl may be optionally substituted by one or more hydroxy or aryl; and wherein any aryl group may be optionally substituted by one hydroxy.

R² is selected from C₁₋₆alkyl; wherein said C₁₋₆alkyl may be optionally substituted by one or more hydroxy or aryl.

R² is selected from C₁₋₆alkyl, C₃₋₆cycloalkyl or phenyl; wherein said C₁₋₆alkyl may be optionally substituted by one or more hydroxy, amino, guanidino, carboxy, carbamoyl, C₁₋₆alkylS(O)ₐ wherein a is 0, C₃₋₆cycloalkyl, phenyl, imidazolyl or indolyl; and wherein any aryl group may be optionally substituted by one substituent selected from hydroxy.

R² is selected from C₁₋₆alkyl; wherein said C₁₋₆alkyl may be optionally substituted by one or more hydroxy or phenyl.

R² is selected from methyl, isopropyl, isobutyl, hydroxymethyl, carboxymethyl, carbamoylmethyl, 2-carboxyethyl, 2-hydroxyethyl, 2-methylthioethyl, 4-aminobutyl, cyclohexylmethyl, benzyl, indol-3-ylmethyl, imidazol-4-ylmethyl, 4-hydroxybenzyl, cyclohexyl, phenyl, 4-hydroxyphenyl or 4-guinadinophenyl.

R² is selected from hydroxymethyl, isobutyl or benzyl.

R³ is hydrogen or halo.

R³ is hydrogen or fluoro.

R³ is fluoro.

R³ is hydrogen.

R⁴ is hydrogen or halo.

R⁴ is hydrogen or fluoro.

R⁴ is fluoro.

R⁴ is hydrogen.

Therefore in a further aspect of the invention, there is provided a compound of formula (I) (as depicted above) wherein:

R¹ is selected from hydrogen, C₁₋₆alkyl or aryl wherein said C₁₋₆alkyl may be optionally substituted by aryl;

R² is selected from C₁₋₆alkyl, C₃₋₆cycloalkyl or aryl; wherein said C₁₋₆alkyl may be optionally substituted by one or more hydroxy, amino, guanidino, carboxy, carbamoyl, C₁₋₆alkylS(O)ₐ wherein a is 0, C₃₋₆cycloalkyl or aryl; and wherein any aryl group may be optionally substituted by one substituent selected from hydroxy;

R³ is hydrogen or halo;

R⁴ is hydrogen or halo;

or a pharmaceutically acceptable salt, solvate, solvate of such a salt or a prodrug thereof.

Therefore in a further aspect of the invention, there is provided a compound of formula (I) (as depicted above) wherein:

R¹ is selected from isobutyl or benzyl;

R² is selected from hydroxymethyl, isobutyl or benzyl;

R³ is fluoro;

R⁴ is fluoro;

or a pharmaceutically acceptable salt, solvate, solvate of such a salt or a prodrug thereof.

In a further aspect of the invention there is provided a compound of formula (I) (as depicted above) wherein:

R¹ is selected from hydrogen, C₁₋₆alkyl, aryl or benzyl;

R² is selected from hydrogen, C₁₋₆alkyl or aryl; wherein said C₁₋₆alkyl may be optionally substituted by one or more hydroxy, C₁₋₆alkoxy or aryl; and wherein any aryl group may be optionally substituted by one hydroxy;

R³ is hydrogen or halo;

R⁴ is hydrogen or halo;

or a pharmaceutically acceptable salt, solvate, solvate of such a salt or a prodrug thereof.

In another aspect of the invention, preferred compounds of the invention are any one of the examples or a pharmaceutically acceptable salt, solvate, solvate of such a salt or a prodrug thereof.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt, solvate, solvate of such a salt or a prodrug thereof which process (wherein variable groups are, unless otherwise specified, as defined in formula (I)) comprises of:
Process 1) Reacting a Compound of Formula (II): 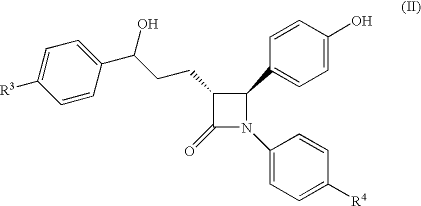
with a compound of formula (III): 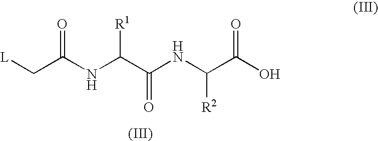
wherein L is a displaceable group;
Process 2) Reacting an Acid of Formula (IV): 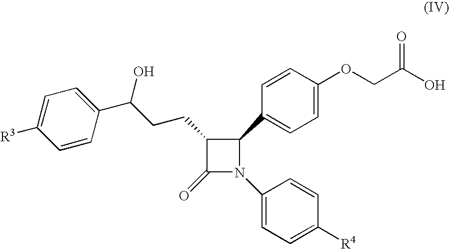
or an activated derivative thereof; with an amine of formula (V): 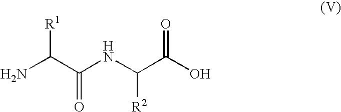
Process 3): Reacting an Acid of Formula (VI): 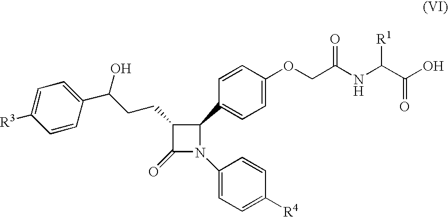
or an activated derivative thereof, with an amine of formula (VII): 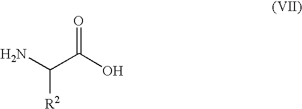
Process 4): Reducing a Compound of Formula (VIII): 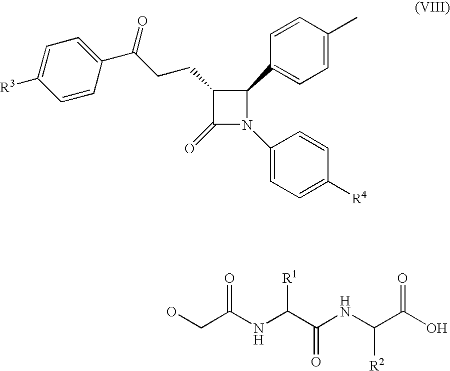
Process 5): De-Esterifying a Compound of Formula (IX) 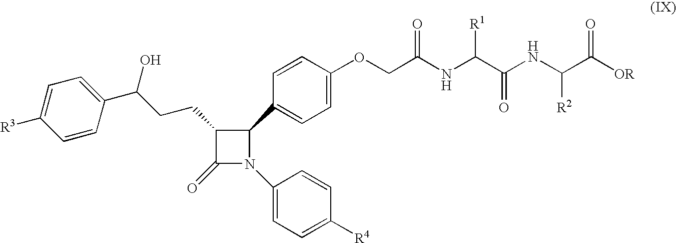
wherein the group C(O)OR is an ester group;
and thereafter if necessary or desirable:
i) converting a compound of the formula (I) into another compound of the formula (I);
ii) removing any protecting groups;
iii) forming a pharmaceutically acceptable salt, solvate, solvate of such a salt or a prodrug; or
iv) separating two or more enantiomers.

L is a displaceable group, suitable values for L are for example, a halogeno or sulphonyloxy group, for example a chloro, bromo, methanesulphonyloxy or toluene-4-sulphonyloxy group.

C(O)OR is an ester group, suitable values for C(O)OR are methoxycarbonyl, ethoxycarbonyl, t-butoxycarbonyl and benzyloxycarbonyl.

The compound of the formula (VI) is an intermediate in the process of preparing formula (I).

The starting materials used in the present invention can be prepared by modifications of the routes described in EP 0 792 264 B1. Alternatively they can be prepared by the following reactions.

Process 1): Alcohols of formula (II) may be reacted with compounds of formula (III) in the presence of a base for example an inorganic base such as sodium carbonate, or an organic base such as Hunigs base, in the presence of a suitable solvent such as acetonitrile, dichloromethane or tetrahydrofuran at a temperature in the range of 0° C. to reflux, preferably at or near reflux.

Compounds of formula (II) may be prepared according to the following scheme:

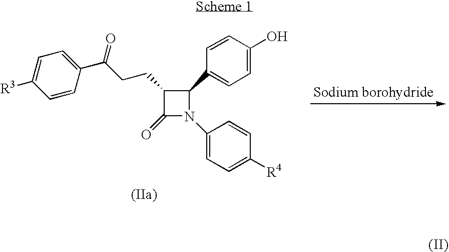

Compounds of formula (IIa) may be prepared according to the procedure, or by analogy with the procedure described in Guangzhong Wu, YeeShing Wong, Xing Chen and Zhixian Ding, J. Org. Chem. 1999, 64, 3714.

Process 2) and Process 3): Acids and amines may be coupled together in the presence of a suitable coupling reagent. Standard peptide coupling reagents known in the art can be employed as suitable coupling reagents, for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Acids of formula (IV) and (VI) may be prepared from compounds of formula (II) by reacting them with the appropriate, optionally protected, side chain using the conditions of Process 1).

Amines of formula (V) and (VII) are commercially available compounds, or they are known in the literature, or they are prepared by standard processes known in the art. Process 4): Reduction of compounds of formula (VIII) could be performed with a hydride reagent such as sodium borohydride in a solvent such as methanol at temperatures suitable between −20-40° C.

Compounds of formula (VIII) can be prepared from compounds of formula (IIa), by performing Process 1.

Process 5): Esters of formula (IX) may be deprotected under standard conditions such as those described below, for example a methyl or ethyl ester may be deprotected with sodium hydroxide in methanol at room temperature.

Compounds of formula (IX) may be prepared from compounds of formula (II) by reacting them with the appropriate protected side chain using the conditions of Process 1).

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess cholesterol absorption inhibitory activity. These properties may be assessed, using the following biological tests.

In Vivo Testing of Cholesterol Absorption Inhibitors (A)

C57BL/6 female mice were maintained on regular chow diet and housed in individual cages to collect faeces. Mice were fasted for 3 hours and then gavaged with vehicle or compound. Half an hour later the mice were gavaged with radiolabelled cholesterol. Six hours after the ¹⁴C-cholesterol gavage blood samples were taken via the tail and plasma prepared to determine how much cholesterol were absorbed. 24 hours after the gavage of ¹⁴C-cholesterol the mice were bled and plasma were prepared for analysis. Faeces were collected for 24 hours to assess absorption efficiency.

In Vivo Testing of Cholesterol Absorption Inhibitors (B).

C57BL/6 female mice were maintained on regular chow diet and housed in individual cages to collect faeces. Mice were fasted for 3 hours and then gavaged with vehicle or compound. One to ten hours later the mice were gavaged with radiolabelled cholesterol. Six hours after the ¹⁴C-cholesterol gavage blood sample was taken via the tail and plasma prepared to determine how much cholesterol was absorbed. 24 hours after the gavage of ¹⁴C-cholesterol the mice were bled and plasma analysed for radioactivity. Faeces were also collected for 24 hours to assess absorption efficiency.

### REFERENCES

1. E. A. Kirk, G. L. Moe, M. T. Caldwell, J. A. Lermark, D. L. Wilson, R. C. LeBoeuf. Hyper- and hypo-responsiveness to dietary fat and cholesterol among inbred mice: searching for level and variability genes. J. Lipid Res. 1995 36:1522-1532.
2. C. P. Carter, P. N. Howles, D. Y. Hui. Genetic variation in cholesterol absorption efficiency among inbred strains of mice. J. Nutr. 1997 127:1344-1348.
3. C. D. Jolley, J. M. Dietschy, S. D. Turley. Genetic differences in cholesterol absorption in 129/Sv and C57BL/6 mice: effect on cholesterol responsiveness. Am. J. Physiol. 1999 276:G1117-G1124.

Administration of 5 μmol/kg of Example 3 gave 75% inhibition of ¹⁴C-cholesterol absorption (procedure A). Administration of 5 μmol/kg of Example 4 gave 58% inhibition of ¹⁴C-cholesterol absorption (procedure A).

Absorption

The absorption of the compounds of formula (I) can be tested in a Caco-2 cells model (Gastroenterology 1989, 96,736).

According to a further aspect of the invention there is provided a pharmaceutical composition which comprises a compound of formula (I), or a pharmaceutically acceptable salt, solvate, solvate of such a salt or a prodrug thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I), or a pharmaceutically acceptable salt, solvate, solvate of such a salt or a prodrug thereof, will normally be administered to a warm-blooded animal at a unit dose within the range of approximately 0.02-100 mg/kg, preferably 0.02 -50 mg/kg, and this normally provides a therapeutically-effective dose. A unit dose form such as a tablet or capsule will usually contain, for example 1-250 mg of active ingredient. Preferably a daily dose in the range of 1-50 mg/kg, particularly 0.1-10 mg/kg is employed. In another aspect a daily dose in the rage of 0.01-20 mg/kg is employed. In one aspect of the invention the daily dose of a compound of formula (I) is less than or equal to 100 mg. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt, solvate, solvate of such a salt or a prodrug thereof, as defined hereinbefore for use in a method of prophylactic or therapeutic treatment of a warm-blooded animal, such as man.

We have found that the compounds defined in the present invention, or a pharmaceutically acceptable salt, solvate, solvate of such a salt or a prodrug thereof, are effective cholesterol absorption inhibitors, and accordingly have value in the treatment of disease states associated with hyperlipidaemic conditions.

Thus according to this aspect of the invention there is provided a compound of the formula (I), or a pharmaceutically acceptable salt, solvate, solvate of such a salt or a prodrug thereof, as defined hereinbefore for use as a medicament.

According to another feature of the invention there is provided the use of a compound of the formula (I), or a pharmaceutically acceptable salt, solvate, solvate of such a salt or a prodrug thereof, as defined hereinbefore in the manufacture of a medicament for use in the production of a cholesterol absorption inhibitory effect in a warm-blooded animal, such as man.

According to another feature of the invention there is provided the use of a compound of the formula (I), or a pharmaceutically acceptable salt, solvate, solvate of such a salt or a prodrug thereof, as defined hereinbefore in the production of a cholesterol absorption inhibitory effect in a warm-blooded animal, such as man.

Herein, where the production of a cholesterol absorption inhibitory effect or a cholesterol lowering effect is stated, suitably this relates to the treatment of hyperlipidaemic conditions in a warm-blooded animal, such as man. Additionally is relates to the treatment of dyslipidemic conditions and disorders such as hyperlipidaemia, hypertrigliceridemia, hyperbetalipoproteinemia (high LDL), hyperprebetalipoproteinemia (high VLDL), hyperchylomicronemia, hypolipoproteinemia, hypercholesterolemia, hyperlipoproteinemia and hypoalphalipoproteinemia (low HDL) in a warm-blooded animal, such as man. Furthermore it relates to the treatment of different clinical conditions such as atherosclerosis, arteriosclerosis, arrhythmia, hyper-thrombotic conditions, vascular dysfunction, endothelial dysfunction, heart failure, coronary heart diseases, cardiovascular diseases, myocardial infarction, angina pectoris, peripheral vascular diseases, inflammation of cardiovascular tissues such as heart, valves, vasculature, arteries and veins, aneurisms, stenosis, restenosis, vascular plaques, vascular fatty streaks, leukocytes, monocytes and/or macrophage infiltration, intimal thickening, medial thinning, infectious and surgical trauma and vascular thrombosis, stroke and transient ischaemic attacks in a warm-blooded animal, such as man. It also relates to the treatment of atherosclerosis, coronary heart diseases, myocardial infarction, angina pectoris, peripheral vascular diseases, stroke and transient ischaemic attacks in a warm-blooded animal, such as man.

The production of a cholesterol absorption inhibitory effect or a cholesterol lowering effect also relates to a method of treating and/or preventing atherosclerotic lesions, a method of preventing plaque rupture and a method of promoting lesion regression. Furthermore it relates to a method of inhibiting monocytes-macrophage accumulation in atherosclerotic lesions, a method of inhibiting expression of matrix metalloproteinases in atherosclerotic lesions, a method of inhibiting the destabilization of atherosclerotic lesions, a method for preventing atherosclerotic plaque rupture and a method of treating unstable angina.

The production of a cholesterol absorption inhibitory effect or a cholesterol lowering effect also relates to a method of treating sitosterolemia.

Compounds of formula (I), or a pharmaceutically acceptable salt, solvate, solvate of such a salt or a prodrug thereof may also have value in the treatment or prevention of Alzeheimer's Disease (see for example WO 02/096415). Therefore in a further aspect of the invention, there is provided a compound of formula (I), or a pharmaceutically acceptable salt, solvate, solvate of such a salt or a prodrug thereof, for use in the treatment or prevention of Alzeheimer's Disease.

Compounds of formula (I), or a pharmaceutically acceptable salt, solvate, solvate of such a salt or a prodrug thereof may also have value in the treatment or prevention of vascular inflammation (see for example WO 03/026644). Therefore in a further aspect of the invention, there is provided a compound of formula (I), or a pharmaceutically acceptable salt, solvate, solvate of such a salt or a prodrug thereof, for use in the treatment or prevention of vascular inflammation.

According to a further feature of this aspect of the invention there is provided a method for producing a cholesterol absorption inhibitory effect in a warm-blooded animal, such as man, in need of such treatment which comprises administering to said animal an effective amount of a compound of formula (I), or a pharmaceutically acceptable salt, solvate, solvate of such a salt or a prodrug thereof.

The cholesterol absorption inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. According to this aspect of the invention there is provided a pharmaceutical product comprising a compound of the formula (I), or a pharmaceutically acceptable salt, solvate, solvate of such a salt or a prodrug thereof, as defined hereinbefore and an additional cholesterol absorption inhibitory substance as defined hereinbefore and an additional hypolipidaemic agent for the conjoint treatment of hyperlipidaemia.

In another aspect of the invention, the compound of formula (I), or a pharmaceutically acceptable salt, solvate, solvate of such a salt or a prodrug thereof, may be administered in association with cholesterol biosynthesis inhibitors, or pharmaceutically acceptable salts, solvates, solvates of such salts or prodrugs thereof. Suitable cholesterol biosynthesis inhibitors include HMG Co-A reductase inhibitors, squalene synthesis inhibitors and squalene epoxidase inhibitors. A suitable squalene synthesis inhibitor is squalestatin 1 and a suitable squalene epoxidase inhibitor is NB-598.

In this aspect of the invention, the compound of formula (I), or a pharmaceutically acceptable salt, solvate, solvate of such a salt or a prodrug thereof, may be administered in association with an HMG Co-A reductase inhibitor, or pharmaceutically acceptable salts, solvates, solvates of such salts or prodrugs thereof. Suitable HMG Co-A reductase inhibitors, pharmaceutically acceptable salts, solvates, solvates of such salts or prodrugs thereof are statins well known in the art. Particular statins are fluvastatin, lovastatin, pravastatin, simvastatin, atorvastatin, cerivastatin, bervastatin, dalvastatin, mevastatin and rosuvastatin, or a pharmaceutically acceptable salt, solvate, solvate of such a salt or a prodrug thereof. A further particular statin is pitvastatin, or a pharmaceutically acceptable salt, solvate, solvate of such a salt or a prodrug thereof. A particular statin is atorvastatin, or a pharmaceutically acceptable salt, solvate, solvate of such a salt or a prodrug thereof. A more particular statin is atorvastatin calcium salt. A further particular statin is rosuvastatin, or a pharmaceutically acceptable salt, solvate, solvate of such a salt or a prodrug thereof. A preferable particular statin is rosuvastatin calcium salt.

Therefore in an additional feature of the invention, there is provided a combination of compound of formula (I), or a pharmaceutically acceptable salt, solvate, solvate of such a salt or a prodrug thereof and an HMG Co-A reductase inhibitor, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of an HMG Co-A reductase inhibitor, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof.

According to a further aspect of the present invention there is provided a kit comprising:
a) a compound of formula (I), or a pharmaceutically acceptable salt, solvate, solvate of such a salt or a prodrug thereof, in a first unit dosage form;
b) an HMG Co-A reductase inhibitor, or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:
a) a compound of formula (I), or a pharmaceutically acceptable salt, solvate, solvate of such a salt or a prodrug thereof, together with a pharmaceutically acceptable diluent or carrier, in a first unit dosage form;
b) an HMG Co-A reductase inhibitor, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in the manufacture of a medicament for use in the production of a cholesterol lowering effect.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of an HMG Co-A reductase inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of a matrix metalloproteinase inhibitor.

In another aspect of the invention, the compound of formula (I), or a pharmaceutically acceptable salt, solvate, solvate of such a salt or a prodrug thereof, may be administered in association with an ileal bile acid (IBAT) inhibitor or a pharmaceutically acceptable salt, solvate, solvate of such a salt or a prodrug thereof. Suitable compounds possessing IBAT inhibitory activity for use in combination with compounds of the present invention have been described, see for instance the compounds described in WO 93/16055, WO 94/18183, WO 94/18184, WO 94/24087, WO 96/05188, WO 96/08484, WO 96/16051, WO 97/33882, WO 98/07749, WO 98/38182, WO 98/40375, WO 98/56757, WO 99/32478, WO 99/35135, WO 99/64409, WO 99/64410, WO 00/01687, WO 00/20392, WO 00/20393, WO 00/20410, WO 00/20437, WO 00/35889, WO 01/34570, WO 00/38725, WO 00/38726, WO 00/38727, WO 00/38728, WO 00/38729, WO 00/47568, WO 00/61568, WO 01/66533, WO 01/68096, WO 01/68637, WO 02/08211, DE 19825804, JP 10072371, US 5070103, EP 251 315, EP 417 725, EP 489 423, EP 549 967, EP 573 848, EP 624 593, EP 624 594, EP 624 595, EP 864 582, EP 869 121 and EP 1 070 703 and the contents of these patent applications are incorporated herein by reference. Particularly the named examples of these patent applications are incorporated herein by reference. More particularly claim 1 of these patent application are incorporated herein by reference.

Other suitable classes of IBAT inhibitors for use in combination with compounds of the present invention are the 1,2-benzothiazepines, 1,4-benzothiazepines and 1,5-benzothiazepines. A further suitable class of IBAT inhibitors is the 1,2,5-benzothiadiazepines.

One particular suitable compound possessing IBAT inhibitory activity for use in combination with compounds of the present invention is (3R,5R)-3-butyl-3-ethyl-1,1-dioxido-5-phenyl-2,3,4,5-tetrahydro-1,4-benzothiazepin-8-yl-3-D-glucopyranosiduronic acid (EP 864 582).

A further suitable compound possessing IBAT inhibitory activity for use in combination with compounds of the present invention is S-8921 (EP 597 107).

A further suitable IBAT inhibitor for use in combination with compounds of the present invention is the compound:

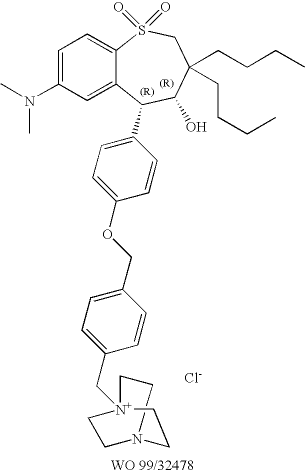

A particular IBAT inhibitor for use in combination with compounds of the present invention is selected from any one of Examples 1-120 of WO 02/50051, or a pharmaceutically acceptable salt, solvate, solvate of such a salt or a prodrug thereof, and the compounds of Examples 1-120 are incorporated herein by reference. Claims 1-15 of WO 02/50051 are also incorporated herein by reference. A particular IBAT inhibitor selected from WO 02/50051 for use in combination with compounds of the present invention is selected from any one of:
1,1-dioxo-3,3-dibutyl-5-phenyl-7-methylthio-8-(N—{(R)-1′-phenyl-1′-[N′-(carboxymethyl)carbamoyl]methyl }carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(carboxymethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N—{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N═-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(2-sulphoethyl)carbamoyl]-4-hydroxybenzyl lcarbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(2-carboxyethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N═-(5-carboxypentyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(R)-(2-hydroxy-1-carboxyethyl)carbamoyl]benzyl }carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(R)-(2-hydroxy-1-carboxyethyl)carbamoyl]benzyl }carbamoylmethoxy)-2,3,4,5-tetrahydro-1,5-benzothiazepine;
1,1-dioxo-3,3-dibutyl-5-phenyl-7-methylthio-8-{N—[(R)-α-(N′—{(R)-1-[N″—(R)-(2-hydroxy-1-carboxyethyl)carbamoyl]-2-hydroxyethyl }carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N′-(carboxymethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphoryl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N-[(R)-α-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N—{(R)-α-[N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N—[(R)-α-(N′-{2-[(methyl)(ethyl)phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N—[(R)-α-(N′-{2-[(methyl)(hydroxy)phosphoryl]ethyl}carbamoyl)4-hydroxybenzyl]carbamoylmethoxy}-2,3,4,5-tetrahydro-1,5-benzothiazepine;
1,1-dioxo-3,3-dibutyl-5-phenyl-7-methylthio-8-(N—{(R)-α-[(R)—N′-(2-methylsulphinyl-1-carboxyethyl)carbamoyl]benzyl}carbamoylmethoxy)-2,3,4,5-tetrahydro-1,5-benzothiazepine; and
1,1-dioxo-3,3-dibutyl-5-phenyl-7-methoxy-8-[N—{(R)-α-[N′-(2-sulphoethyl)carbamoyl]-4-hydroxybenzyl}carbamoylmethoxy)-2,3,4,5-tetrahydro-1,5-benzothiazepine;
   or a pharmaceutically acceptable salt, solvate, solvate of such a salt or a prodrug thereof.

A particular IBAT inhibitor for use in combination with compounds of the present invention is selected from any one of Examples 1-44 of WO 03/020710, or a pharmaceutically acceptable salt, solvate, solvate of such a salt or a prodrug thereof, and the compounds of Examples 1-44 are incorporated herein by reference. Claims 1-10 of WO 03/020710 are also incorporated herein by reference. A particular IBAT inhibitor selected from WO 03/020710 for use in combination with compounds of the present invention is selected from any one of:
1,1-dioxo-3,3-dibutyl-5-phenyl-7-methylthio-8-(N—{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-Ε-[N′-(2-(S)-3-(R)-4-(R)-5-(R)-2,3,4,5,6-pentahydroxyhexyl)carbamoyl]benzyl carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-((S)-1-carbamoyl-2-hydroxyethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(hydroxycarbamoyl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-[N—((R)-α-{N′-[2-(N′-pyrimidin-2-ylureido)ethyl]carbamoyl}benzyl)carbamoylmethoxy]-2,3,4,5-tetrahydro-1,5-benzothiazepine;
1,1-dioxo-3-butyl-3-ethyl-5-phenyl-7-methylthio-8-[N—((R)-α-{N′-[2-(N′-pyridin-2-ylureido)ethyl]carbamoyl}benzyl)carbamoylmethoxy]-2,3,4,5-tetrahydro-1,5-benzothiazepine;
1,1-dioxo-3-butyl-3-ethyl-5-phenyl-7-methylthio-8-(N—{(R)-α-[N′-(1-t-butoxycarbonylpiperidin-4-ylmethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(2,3-dihydroxypropyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-[N—((R)-α-{N′-[2-(3,4-dihydroxyphenyl)-2-methoxyethyl]carbamoyl}benzyl)carbamoylmethoxy]-2,3,4,5-tetrahydro-1,5-benzothiazepine
1,1-dioxo-3-butyl-3-ethyl-5-phenyl-7-methylthio-8-(N—{(R)-α-[N′-(2-aminoethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(piperidin-4-ylmethyl)carbamoyl]benzyl}carbamoylmethoxy)-2,3,4,5-tetrahydro-1,5-benzothiazepine; or
1,1-dioxo-3-butyl-3ethyl-5-phenyl-7-methylthio-8-(N—{(R)-α-[N′-(2-N,N-dimethylaminosulphamoylethyl)carbamoyl]benzyl}carbamoylmethoxy)-2,3,4,5-tetrahydro-1,5-benzothiazepine;
   or a pharmaceutically acceptable salt, solvate, solvate of such a salt or a prodrug thereof.

A particular IBAT inhibitor for use in combination with compounds of the present invention is selected from any one of Examples 1-7 of WO 03/022825, or a pharmaceutically acceptable salt, solvate, solvate of such a salt or a prodrug thereof, and the compounds of Examples 1-7 are incorporated herein by reference. Claims 1-8 of WO 03/022825 are also incorporated herein by reference. A particular IBAT inhibitor selected from WO 03/022825 for use in combination with compounds of the present invention is selected from any one of:
1,1-dioxo-3(R)-3-butyl-3-ethyl-5-(R)-5-phenyl-8-[N—((R)-α-carboxybenzyl)carbamoylmethoxy]-2,3,4,5-tetrahydro-1,4-benzothiazepine;
1,1-dioxo-3(S)-3-butyl-3-ethyl-5-(S)-5-phenyl-8-[N-((R)-α-carboxybenzyl)carbamoylmethoxy]-2,3,4,5-tetrahydro-1,4-benzothiazepine;
1,1-dioxo-3(R)-3-butyl-3-ethyl-5-(R)-5-phenyl-8-(N—{(R)-α-[N-(carboxymethyl)carbamoyl]benzyl}carbamoylmethoxy)-2,3,4,5-tetrahydro-1,4-benzothiazepine;
1,1-dioxo-3(S)-3-butyl-3-ethyl-5-(S)-5-phenyl-8-(N—{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-bromo-8-(N—{(R)-α-[N-(carboxymethyl)carbamoyl]benzyl}carbamoylmethoxy)-2,3,4,5-tetrahydro-1,4-benzothiazepine;
3,5-trans-1,1-dioxo-3-(S)-3-ethyl-3-butyl-4-hydroxy-5-(S)-5-phenyl-7-bromo-8-(N—{(R)-α-[N-(carboxymethyl)carbamoyl]benzyl}carbamoylmethoxy)-2,3,4,5-tetrahydro-1,4-benzothiazepine
3,5-trans-1,1-dioxo-3-(R)-3-ethyl-3-butyl-4-hydroxy-5-(R)-5-phenyl-7-bromo-8-(N—{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-methylthio-8-(N—{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-methylthio-8-(N—{(R)-α-[N-(2-sulphoethyl)carbamoyl]-4-hydroxybenzyl}carbamoylmethoxy)-2,3,4,5-tetrahydro-1,4-benzothiazepine ammonia salt;
1,1-dioxo-3-(S)-3-ethyl-3-butyl-5-(S)-5-phenyl-7-methylthio-8-(N—{(R)-α-[N-(carboxymethyl)carbamoyl]benzyl}carbamoylmethoxy)-2,3,4,5-tetrahydro-1,4-benzothiazepine diethylamine salt; and
1,1-dioxo-3-(R)-3-ethyl-3-butyl-5-(R)-5-phenyl-7-methylthio-8-(N—{(R)-α-[N-(carboxymethyl)carbamoyl]benzyl}carbamoylmethoxy)-2,3,4,5-tetrahydro-1,4-benzothiazepine diethylamine salt;
   or a pharmaceutically acceptable salt, solvate, solvate of such a salt or a prodrug thereof.

A particular IBAT inhibitor for use in combination with compounds of the present invention is selected from any one of Examples 1-4 of WO 03/022830, or a pharmaceutically acceptable salt, solvate, solvate of such a salt or a prodrug thereof, and the compounds of Examples 1-4 are incorporated herein by reference. Claims 1-8 of WO 03/022830 are also incorporated herein by reference. A particular IBAT inhibitor selected from WO 03/022830 for use in combination with compounds of the present invention is selected from any one of:
1,1-dioxo-3-butyl-3-ethyl-4-hydroxy-5-phenyl-7-(N—{(R)-α-[N-(carboxymethyl)carbamoyl]benzyl}carbamoylmethylthio)-2,3,4,5-tetrahydrobenzothiepine
1,1-dioxo-3-butyl-3-ethyl-4-hydroxy-5-phenyl-7-(N—{(R)-α-[N-(2-sulphoethyl)carbamoyl]hydroxybenzyl}carbamoylmethylthio)-2,3,4,5-tetrahydrobenzothiepine ammonia salt
1,1-dioxo-3-butyl-3-ethyl-4-hydroxy-5-phenyl-7-{N-[α-(carboxy)-2-fluorobenzyl]carbamoylmethylthio}-2,3,4,5-tetrahydrobenzothiepine; and
1,1-dioxo-3-butyl-3-ethyl-4-hydroxy-5-phenyl-7-{N-[1-(carboxy)-1-(thien-2-yl)methyl]carbamoylmethylthio}-2,3,4,5-tetrahydrobenzothiepine
   or a pharmaceutically acceptable salt, solvate, solvate of such a salt or a prodrug thereof.

A particular IBAT inhibitor for use in combination with compounds of the present invention is selected from any one of Examples 1-39 of WO 03/022286, or a pharmaceutically acceptable salt, solvate, solvate of such a salt or a prodrug thereof, and the compounds of Examples 1-39 are incorporated herein by reference. Claims 1-10 of WO 03/022286 are also incorporated herein by reference. A particular IBAT inhibitor selected from WO 03/022286 for use in combination with compounds of the present invention is selected from any one of:
1,1-dioxo-3,3-dibutyl-5-phenyl-7-methylthio-8-(N—{(R)-α-[N-((R)-1-carboxy-2-methylthio-ethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxybutyl)carbamoyl]hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxyethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxyethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((R)-1-carboxy-2-methylthioethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-{(S)-1-[N—((S)-2-hydroxy-1-carboxyethyl)carbamoyl]propyl}carbamoyl]benzyl lcarbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxy-2-methylpropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-((S)-1-carboxypropyl)carbamoyl]-4-hydroxybenzyl}carbamoylmethoxy)-2,3,4,5-tetrahydro-1,2,5-benzothiadiazepine; and
1,1-dioxo-3,3-dibutyl-5-phenyl-7-methylthio-8-[N—((R)-α-carboxy-4-hydroxybenzyl)carbamoylmethoxy]-2,3,4,5-tetrahydro-1,2,5-benzothiadiazepine;
   or a pharmaceutically acceptable salt, solvate, solvate of such a salt or a prodrug thereof.

A particular IBAT inhibitor for use in combination with compounds of the present invention is selected from any one of Examples 1-7 of WO 03/091232, or a pharmaceutically acceptable salt, solvate, solvate of such a salt or a prodrug thereof, and the compounds of Examples 1-7 are incorporated herein by reference. Claims 1-10 of WO 03/091232 are also incorporated herein by reference. A particular IBAT inhibitor selected from WO 03/091232 for use in combination with compounds of the present invention is selected from any one of:
1,1-Dioxo-3,3-dibutyl-5-phenyl-7-methylthio-8-(N—{(R)-α-[N-(2-(S)-3-(R)-4-(R)-5-(R)-2,3,4,5,6-pentahydroxyhexyl)carbamoyl]benzyl}carbamoylmethoxy)-2,3,4,5-tetrahydro-1,2,5-benzothiadiazepine;
1,1-Dioxo-3,3-dibutyl-5-phenyl-7-methylthio-8-(N-{(R)-α-[N-(2-(S)-3-(R)-4-(R)-5-(R)-2,3,4,5,6-pentahydroxyhexyl)carbamoyl]-4-hydroxybenzyl}carbamoylmethoxy)-2,3,4,5-tetrahydro-1,2,5-benzothiadiazepine;
1,1-Dioxo-3,3-dibutyl-5-phenyl-7-methylthio-8-[N—((R/S)-α-{N-[1-(R)-2-(S)-1-hydroxy-1-(3,4-dihydroxyphenyl)prop-2-yl]carbamoyl}-4-hydroxybenzyl)carbamoylmethoxy]-2,3,4,5-tetrahydro-1,2,5-benzothiadiazepine;
1,1-Dioxo-3,3-dibutyl-5-phenyl-7-methylthio-8-{N—[(R)-α-(N-{2-(S)-[N-(carbamoylmethyl)carbamoyl]pyrrolidin-1-ylcarbonylmethyl}carbamoyl)benzyl]carbamoylmethoxy}-2,3,4,5-tetrahydro-1,2,5-benzothiadiazepine;
1,1-Dioxo-3,3-dibutyl-5-phenyl-7-methylthio-8-[N—((R)-α-(N-[2-(3,4,5-trihydroxyphenyl)ethyl]carbamoyl}benzyl)carbamoylmethoxy]-2,3,4,5-tetrahydro-1,2,5-benzothiadiazepine; and
1,1-Dioxo-3,3-dibutyl-5-phenyl-7-methylthio-8-(N—{(R)-α-[N-(2-(R)-3-(S)-4-(S)-5-(R)-3,4,5,6-tetrahydroxytetrahydropyran-2-ylmethyl)carbamoyl]benzyl}carbamoylmethoxy)-2,3,4,5-tetrahydro-1,2,5-benzothiadiazepine;
   or a pharmaceutically acceptable salt, solvate, solvate of such a salt or a prodrug thereof.

Further suitable compounds possessing IBAT inhibitory activity for use in combination with compounds of the present invention is disclosed in WO 03/106482.

Suitable IBAT disclosed in WO 03/106482 for use in combination with compounds, of the present invention are selected from any one of:
1,1-dioxo-3,3-dibutyl-5-phenyl-7-methylthio-8-(N—{(R)-α-[N′-((S)-1-carboxyethyl)carbamoyl]benzyl}carbamoylmethoxy)-2,3,4,5-tetrahydro-1,5-benzothiazepine;
1,1-dioko-3,3-dibutyl-5-phenyl-7-methylthio-8-(N—{(R)-α-[N′-((S)-1-carboxypropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxybutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-methylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-methylbutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-3-methylbutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-((S)-1-carboxy-2-hydroxypropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-2-mesylethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-3-methylsulphonylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-3-mesylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxypropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxybutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-2-methylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-2-methylbutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-3-methylbutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-2-hydroxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-2-hydroxypropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-2-methylthioethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-2-methylsulphinylethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-2-mesylethyl)carbamoyl]-4-hydroxybenzyl}carbamoylmethoxy)-2,3,4,5-tetrahydro 1,5-benzothiazepine;
1,1-dioxo-3,3-dibutyl-5-phenyl-7-methylthio-8-(N—{(R)-α-[N′—((S)-1-carboxy-2-methoxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-3-methylthiopropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-3-methylsulphonylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxy-3-mesylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—((S)-1-carboxypropyl)carbamoyl]-4-hydroxybenzyl}carbamoylmethoxy)-2,3,4,5-tetrahydro-1,5-benzothiazepine; or
1,1-dioxo-3,3-dibutyl-5-phenyl-7-methylthio-8-(N—{(R)-α-[N′—(S)-1-carboxyethyl)carbamoyl]benzyl}carbamoylmethoxy)-2,3,4,5-tetrahydro-1,5-benzothiazepine.
   or a pharmaceutically acceptable salt, solvate, solvate of such a salt or a prodrug thereof.

Further suitable IBAT inhibitors for use in combination with compounds of the present invention are those disclosed in WO 04/076430.

In a particular aspect of the invention an IBAT inhibitor or a pharmaceutically acceptable salt, solvate, solvate of such a salt or a prodrug thereof is an IBAT inhibitor or a pharmaceutically acceptable salt thereof.

Therefore in an additional feature of the invention, there is provided a combination of a compound of formula (I), or a pharmaceutically acceptable salt, solvate, solvate of such a salt or a prodrug thereof and an IBAT inhibitor, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of an IBAT inhibitor, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof.

According to a further aspect of the present invention there is provided a kit comprising:
a) a compound of formula (I), or a pharmaceutically acceptable salt, solvate, solvate of such a salt or a prodrug thereof, in a first unit dosage form;
b) an IBAT inhibitor, or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:
a) a compound of formula (I), or a pharmaceutically acceptable salt, solvate, solvate of such a salt or a prodrug thereof, together with a pharmaceutically acceptable diluent or carrier, in a first unit dosage form;
b) an IBAT inhibitor, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and an IBAT inhibitor, or a pharmaceutically acceptable salt, solvate, solvate of such a salt or a prodrug thereof, in the manufacture of a medicament for use in the production of a cholesterol lowering effect in a warm-blooded animal, such as man.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of an IBAT inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

In another aspect of the invention, the compound of formula (I), or a pharmaceutically acceptable salt, solvate, solvate of such a salt or a prodrug thereof, may be administered in association with a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, WO03/051821, WO03/051822, WO03/051826, PCT/GB03/02584, PCT/GB03/02591, PCT/GB03/02598, J Med Chem, 1996, 39, 665, Expert Opinion on Therapeutic Patents, 10 (5), 623-634 (in particular the compounds described in the patent applications listed on page 634) and J Med Chem, 2000, 43, 527 which are all incorporated herein by reference. Particularly a PPAR alpha and/or gamma agonist refers to WY-14643, clofibrate, fenofibrate, bezafibrate, GW 9578, troglitazone, pioglitazone, rosiglitazone, eglitazone, proglitazone, NN622/Ragaglitazar, BMS 298585, BRL-49634, KRP-297, JTT-501, SB 213068, GW 1929, GW 7845, GW 0207, L-796449, L-165041 and GW 2433. Particularly a PPAR alpha and/or gamma agonist refers to (S)-2-ethoxy-3-[4-(2-{4-methanesulphonyloxyphenyllethoxy)phenyl]propanoic acid and pharmaceutically acceptable salts thereof.

Therefore in an additional feature of the invention, there is provided a combination of a compound of formula (I), or a pharmaceutically acceptable salt, solvate, solvate of such a salt or a prodrug thereof and a PPAR alpha and/or gamma agonist, or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a PPAR alpha and/or gamma agonist, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a PPAR alpha and/or gamma agonist, or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula (I), or a pharmaceutically acceptable salt, solvate, solvate of such a salt or a prodrug thereof, and a PPAR alpha and/or gamma agonist, or a pharmaceutically acceptable salt, solvate, solvate of such a salt or a prodrug thereof.

According to a further aspect of the present invention there is provided a kit comprising:
a) a compound of formula (I), or a pharmaceutically acceptable salt, solvate, solvate of such a salt or a prodrug thereof, in a first unit dosage form;
b) a PPAR alpha and/or gamma agonist, or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:
a) a compound of formula (I), or a pharmaceutically acceptable salt, solvate, solvate of such a salt or a prodrug thereof, together with a pharmaceutically acceptable diluent or carrier, in a first unit dosage form;
b) a PPAR alpha and/or gamma agonist, or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula (I), or a pharmaceutically acceptable salt, solvate, solvate of such a salt or a prodrug thereof, and a PPAR alpha and/or gamma agonist, or a pharmaceutically acceptable salt, solvate, solvate of such a salt or a prodrug thereof, in the manufacture of a medicament for use in producing a cholesterol lowering effect in a warm-blooded animal, such as man.

According to a further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an effective amount of a PPAR alpha and/or gamma agonist, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional future aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of a Apo A-1 Mimetic Peptide.

In another aspect of the invention, there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration of an agonists to the receptor HM74A (nicotinic acid receptor). HM74A agonists may be nicotine acid derivates. As used herein “nicotinic acid derivative” means a compounds comprising a pyridine-3-carboxylate structure or a pyrazine-2-carboxylate structure. Examples of nicotinic acid derivatives include nicotinic acid, niceritrol, nicofuranose, NIASPAN® and acipimox.

Therefore, in an additional feature of the invention, there is provided a combination of a compound of formula (I), or a pharmaceutically acceptable salt, solvate, solvate of such a salt or a prodrug thereof and a nicotinic acid derivative or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a nicotinic acid derivative, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a nicotinic acid derivative, or a pharmaceutically acceptable salt, solvate, solvate of such a salt or a prodrug thereof, in association with a pharmaceutically acceptable diluent or carrier.

According to another feature of the invention there is provided the use of a compound of the formula (I), or a pharmaceutically acceptable salt, solvate, solvate of such a salt or a prodrug thereof, and a nicotinic acid derivative, or a pharmaceutically acceptable salt, solvate, solvate of such a salt or a prodrug thereof, in the manufacture of a medicament for use in the production of a cholesterol lowering effect in a warm-blooded animal, such as man.

In another aspect of the invention, the compound of formula (I), or a pharmaceutically acceptable salt, solvate, solvate of such a salt or a prodrug thereof, may be administered in association with a bile acid sequestrant or a pharmaceutically acceptable salt, solvate, solvate of such a salt or a prodrug thereof. Suitable bile acid sequestrants include cholestyramine, cholestipol and cosevelam hydrochloride.

Therefore, in an additional feature of the invention, there is provided a combination of a compound of formula (I), or a pharmaceutically acceptable salt, solvate, solvate of such a salt or a prodrug thereof and a bile acid sequestrant or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a bile acid sequestrant, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a bile acid sequestrant, or a pharmaceutically acceptable salt, solvate, solvate of such a salt or a prodrug thereof, in association with a pharmaceutically acceptable diluent or carrier.

According to another feature of the invention there is provided the use of a compound of the formula (I), or a pharmaceutically acceptable salt, solvate, solvate of such a salt or a prodrug thereof, and a bile acid sequestrant, or a pharmaceutically acceptable salt, solvate, solvate of such a salt or a prodrug thereof, in the manufacture of a medicament for use in the production of a cholesterol lowering effect in a warm-blooded animal, such as man.

According to an additional further aspect of the present invention there is provided a combination treatment comprising the administration of an effective amount of a compound of the formula (I), or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from Group X:
an antihypertensive compound (for example althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyidopa, metoprolol succinate, moexipril hydrochloride, monatepil maleate, pelanserin hydrochloride, phenoxybenzemine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate and bevantolol hydrochloride);
an angiotensin converting enzyme inhibitor (for example alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat);
an angiotensin II receptor antagonist (for example candesartan, candesartan cilexetil, losartan, valsartan, irbesartan, tasosartan, telmisartan and eprosartan);
an andrenergic blocker (for example bretylium tosylate, dihydroergotamine so mesylate, phentolamine mesylate, solypertine tartrate, zolertine hydrochloride, carvedilol or labetalol hydrochloride); an alpha andrenergic blocker (for example fenspiride hydrochloride, labetalol hydrochloride, proroxan and alfuzosin hydrochloride); a beta andrenergic blocker (for example acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate and nebivolol); or a mixed alpha/beta andrenergic blocker;
an andrenergic stimulant (for example combination product of chlorothiazide and methyidopa, the combination product of methyidopa hydrochlorothiazide and methyidopa, clonidine hydrochloride, clonidine, the combination product of chlorthalidone and clonidine hydrochloride and guanfacine hydrochloride);
channel blocker, for example a calcium channel blocker (for example clentiazem maleate, amlodipine besylate, isradipine, nimodipine, felodipine, nilvadipine, nifedipine, teludipine hydrochloride, diltiazem hydrochloride, belfosdil, verapamil hydrochloride or fostedil);
a diuretic (for example the combination product of hydrochlorothiazide and spironolactone and the combination product of hydrochlorothiazide and triamterene);
anti-anginal agents (for example amlodipine besyl ate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochoride, tosifen or verapamil hydrochloride);
vasodilators for example coronary vasodilators (for example fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexiline maleate, prenylamine, propatyl nitrate, terodiline hydrochloride, tolamolol and verapamil);
anti-coagulants (selected from argatroban, bivalirudin, dalteparin sodium, desirudin, dicumarol, Iyapolate sodium, nafamostat mesylate, phenprocoumon, tinzaparin sodium and warfarin sodium);
antithrombotic agents (for example anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab and zolimomab aritox);
fibrinogen receptor antagonists (for example roxifiban acetate, fradafiban, orbofiban, lotrafiban hydrochloride, tirofiban, xemilofiban, monoclonal antibody 7E3 and sibrafiban)
platelet inhibitors (for example cilostezol, clopidogrel bisulfate, epoprostenol, epoprostenol sodium, ticlopidine hydrochloride, aspirin, ibuprofen, naproxen, sulindae, indomethacin, mefenamate, droxicam, diclofenac, sulfinpyrazone and piroxicam, dipyridamole);
platelet aggregation inhibitors (for example acadesine, beraprost, beraprost sodium, ciprostene calcium, itezigrel, lifarizine, lotrafiban hydrochloride, orbofiban acetate, oxagrelate, fradafiban, orbofiban, tirofiban and xemilofiban)
hemorrheologic agents (for example pentoxifylline);
lipoprotein associated coagulation inhibitors;
Factor VIIa inhibitors;
Factor Xa inhibitors;
low molecular weight heparins (for example enoxaparin, nardroparin, dalteparin, certroparin, parnaparin, reviparin and tinzaparin);
squalene synthase inhibitors;
squalene epoxidase inhibitors;
liver X receptor (LXR) agonists for example GW-3965 and those described in WO00224632, WO00103705, WO02090375 and WO00054759 (claim 1 and the named examples of these four application are incorporated herein by reference);
microsomal triglyceride transfer protein inhibitors for example implitapide and those described in WO03004020, WO03002533, WO02083658 and WO 00242291 (claim 1 and the named examples of these four application are incorporated herein by reference);
   or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore, in an additional feature of the invention, there is provided a combination of a compound of formula (I), or a pharmaceutically acceptable salt, solvate, solvate of such a salt or a prodrug thereof and a compound from Group X or a pharmaceutically acceptable salt, solvate, solvate of such a salt or a prodrug thereof.

Therefore in an additional feature of the invention, there is provided a method for producing a cholesterol lowering effect in a warm-blooded animal, such as man, in need of such treatment which comprises administering to said animal an effective amount of a compound of formula (I), or a pharmaceutically acceptable salt, solvate, solvate of such a salt or a prodrug thereof in simultaneous, sequential or separate administration with an effective amount of a compound from Group X, or a pharmaceutically acceptable salt, solvate, solvate of such a salt or a prodrug thereof.

According to a further aspect of the invention there is provided a pharmaceutical composition which comprises a compound of formula (I), or a pharmaceutically acceptable salt, solvate, solvate of such a salt or a prodrug thereof, and a compound from Group X, or a pharmaceutically acceptable salt, solvate, solvate of such a salt or a prodrug thereof, in association with a pharmaceutically acceptable diluent or carrier.

According to another feature of the invention there is provided the use of a compound of the formula (I), or a pharmaceutically acceptable salt, solvate, solvate of such a salt or a prodrug thereof, and a compound from Group X, or a pharmaceutically acceptable salt, solvate, solvate of such a salt or a prodrug thereof, in the manufacture of a medicament for use in the production of a cholesterol lowering effect in a warm-blooded animal, such as man.

In addition to their use in therapeutic medicine, the compounds of formula (I), or a pharmaceutically acceptable salt, solvate, solvate of such a salt or a prodrug thereof, are also useful as pharmacological tools in the development and standardisation of in vitro and in vivo test systems for the evaluation of the effects of inhibitors of cholesterol absorption in laboratory animals such as cats, dogs, rabbits, monkeys, rats and mice, as part of the search for new therapeutic agents.

Many of the intermediates described herein are novel and are thus provided as a further feature of the invention. For example compounds of formula (VI) show cholesterol absorption inhibitory activity when tested in the above referenced in vitro test assay and are thus claimed as a further feature of the invention.

Thus in a further feature of the invention, there is provided a compound of formula (VI), or a pharmaceutically acceptable salt, solvate, solvate of such a salt or a prodrug thereof.

Therefore according to a further aspect of the invention there is provided a pharmaceutical composition which comprises a compound of formula (VI), or a pharmaceutically acceptable salt, solvate, solvate of such a salt or a prodrug thereof in association with a pharmaceutically-acceptable diluent or carrier.

According to an additional aspect of the present invention there is provided a compound of the formula (VI), or a pharmaceutically acceptable salt, solvate, solvate of such a salt or a prodrug thereof, for use in a method of prophylactic or therapeutic treatment of a warm-blooded animal, such as man.

Thus according to this aspect of the invention there is provided a compound of the formula (VI), or a pharmaceutically acceptable salt, solvate, solvate of such a salt or a prodrug thereof, for use as a medicament.

According to another feature of the invention there is provided the use of a-compound of the formula (VI), or a pharmaceutically acceptable salt, solvate, solvate of such a salt or a prodrug thereof, in the manufacture of a medicament for use in the production of a cholesterol absorption inhibitory effect in a warm-blooded animal, such as man.

According to another feature of the invention there is provided the use of a compound of the formula (VI), or a pharmaceutically acceptable salt, solvate, solvate of such a salt or a prodrug thereof, in the manufacture of a medicament for use in the treatment of hyperlipidaemic conditions in a warm-blooded animal, such as man.

According to a further feature of this aspect of the invention there is provided a method for producing a cholesterol absorption inhibitory effect in a warm-blooded animal, such as man, in need of such treatment which comprises administering to said animal, an effective amount of a compound of formula (VI), or a pharmaceutically acceptable salt, solvate, solvate of such a salt or a prodrug thereof.

According to a further feature of this aspect of the invention there is provided a method of treating hyperlipidemic conditions in a warm-blooded animal, such as man, in need of such treatment which comprises administering to said animal an effective amount of a compound of formula (VI), or a pharmaceutically acceptable salt, solvate, solvate of such a salt or a prodrug thereof.

In the above other pharmaceutical composition, process, method, use and medicament manufacture features, the alternative and preferred embodiments of the compounds of the invention described herein also apply.

### EXAMPLES

The invention will now be illustrated in the following non limiting Examples, in which standard techniques known to the skilled chemist and techniques analogous to those described in these Examples may be used where appropriate, and in which, unless otherwise stated:
(i) evaporations were carried out by rotary evaporation in vacuo and work up procedures were carried out after removal of residual solids such as drying agents by filtration;
(ii) all reactions were carried out under an inert atmosphere at ambient temperature, typically in the range 18-25° C., with solvents of HPLC grade under anhydrous conditions, unless otherwise stated;
(iii) column chromatography (by the flash procedure) was performed on Silica gel 40-63 μm (Merck);
(iv) yields are given for illustration only and are not necessarily the maximum attainable;
(v) the structures of the end products of the formula (I) were generally confirmed by nuclear (generally proton) magnetic resonance (NMR) and mass spectral techniques; magnetic resonance chemical shift values were measured in deuterated CDCl₃ (unless otherwise stated) on the delta scale (ppm downfield from tetramethylsilane); proton data is quoted unless otherwise stated; spectra were recorded on a Varian Mercury-300 MHz, Varian Unity plus-400 MHz, Varian Unity plus-600 MHz or on Varian Inova-500 MHz spectrometer unless otherwise stated data was recorded at 400 MHz; and peak multiplicities are shown as follows: s, singlet; d, doublet; dd, double doublet; t, triplet; tt, triple triplet; q, quartet; tq, triple quartet; m, multiplet; br, broad; ABq, AB quartet; ABd, AB doublet, ABdd, AB doublet of doublets; dABq, doublet of AB quartets; LCMS were recorded on a Waters ZMD, LC column xTerra MS C₈(Waters), detection with a HP 1100 MS-detector diode array equipped; mass spectra (MS) (loop) were recorded on VG Platform II (Fisons Instruments) with a HP-1100 MS-detector diode array equipped; unless otherwise stated the mass ion quoted is (MH⁺); unless further details are specified in the text, analytical high performance liquid chromatography (HPLC) was performed on Prep LC 2000 (Waters), Cromasil C₈, 7 μm, (Akzo Nobel); MeCN and de-ionised water 10 mM ammonium acetate as mobile phases, with suitable composition;
(vii) intermediates were not generally fully characterised and purity was assessed by thin layer chromatography (TLC), HPLC, infra-red (IR), MS or NMR analysis;
(viii) where solutions were dried sodium sulphate was the drying agent; and
(ix) the following abbreviations may be used hereinbefore or hereinafter:—
DCM dichloromethane;
TBTU o-Benzotriazol-1-yl-N,N,N′,N′-tetramethyluronium tetrafluoroborate;
EtOAc ethyl acetate; and
MeCN acetonitrile.

### Example 1

### 1-(4-Fluorophenyl)-3-(R)-[3-(4-fluorophenyl)-3-hydroxypropyl]-4-(S)-{4-[N—((R)-1-{N-[1-(S)-(carboxy)-2-(hydroxy)ethyl]carbamoyl}-3-methylbutyl)carbamoylmethoxy]phenyl}azetidin-2-one

1-(4-Fluorophenyl)-3-(R)-[2-(4-fluorobenzoyl)ethyl]-4-(S)-{4-[N—((R)-1-{N-[1-(S)-(t-butoxycarbonyl)-2-(t-butoxy)ethyl]carbamoyl}-3-methylbutyl)carbamoylmethoxy]phenyl}azetidin-2-one (Method 2; 25 mg, 0.032 mmol) was dissolved in formic acid (1 ml) and the mixture was stirred over night at 40-45° C. The formic acid was evaporated and the residue was dissolved in methanol (1 ml). NaBH₄ (5 mg, 0.13 mmol) was added and the mixture was stirred for 15 minutes at room temperature. Two drops of triethylamine was added and the mixture was stirred over night at room temperature. Two drops of acetic acid was added and the mixture was evaporated under reduced pressure. The residue was purified by preparative BPLC using MeCN/ammonium acetate buffer (40:60) as eluent. After freeze-drying 14 mg (66%) of the title compound was obtained. The product was analyzed by LC-MS technique. M/z: 667.7.

### Example 2

### 1-(4-Fluorophenyl)-3-(R)-[3-(4-fluorophenyl)-3-hydroxypropyl]-4-(S)-{4-N—((R)-1-{N-[1-(S)-(carboxy)-3-(methyl)butyl]carbamoyl}-3-methylbutyl)carbamoylmethoxy]phenyl}azetidin-2-one

1-(4-Fluorophenyl)-3-(R)-[2-(4-fluorobenzoyl)ethyl]-4-(S)-{4-[N-((R)-1-{N-[1-(S)-(t-butoxycarbonyl)-3-(methyl)butyl]carbamoyl}-3-methylbutyl)carbamoylmethoxy]phenyl}azetidin-2-one (Method 3; 30 mg, 0.040 mmol) was dissolved in formic acid (1 ml). The mixture was stirred over night at 40-45° C. Formic acid was evaporated under reduced pressure and the residue was dissolved in methanol (1 ml). NaBH (5 mg, 0.13 mmol) was added and the mixture was stirred for 15 minutes at room temperature. Ammonium acetate (20 mg, 0.25 mmol) was added and the mixture was evaporated under reduced pressure. The residue was purified by preparative HPLC using MeCN/ammonium acetate buffer (40:60) as eluent. After freeze-drying 11 mg (40%) of the title compound was obtained. The product was analyzed by LC-MS technique. M/z: 693.8.

### Example 3

### 1-(4-Fluorophenyl)-3-(R)-[3-(4-fluorophenyl)-3-hydroxypropyl]-4-(S)-{4-[N-((R)-1-{N-[1-(R)-(carboxy)-2-(phenyl)ethyl]carbamoyl}-3-methylbutyl)carbamoylmethoxy]phenyl}azetidin-2-one

1-(4-Fluorophenyl)-3-(R)-[2-(4-fluorobenzoyl)ethyl]-4-(S)-{4-[N-((R)-1-{N-[1-(R)-(t-butoxycarbonyl)-2-(phenyl)ethyl]carbamoyl}-3-methylbutyl)carbamoylmethoxy]phenyl}azetidin-2-one (Method 4; 15 mg, 0.019 mmol) was dissolved in formic acid (1 ml) and the mixture was stirred over night at 40-45° C. Formic acid was evaporated under reduced pressure. The residue was dissolved in methanol (0.5 ml) and NaBH₄ (3 mg, 0.079 mmol) was added. The mixture was stirred for 15 minutes at room temperature. Ammonium acetate (20 mg, 0.25 mmol) was added and the solvent was evaporated under reduced pressure. The residue was purified by preparative HPLC using MeCN/ammonium acetate buffer (40:60) as eluent. After freeze-drying 10 mg (72%) of the title compound was obtained. NMR (500 MHz, DMSO): 0.74-0.80 (m, 6H), 1.40-1.51 (m, 3H), 1.67-1.77 (m, 3H), 1.80-1.91 (m, 1H), 2.85-2.95 (m, 1H), 3.00-3.10 (m, 2H), 4.284.38 (m, 2H), 4.41-4.58 (m, 3H), 4.87 (d, 1H), 5.30 (bs, 1H), 6.91-6.95 (m, 2H), 7.10-7.24 (m, 11H), 7.28-7.35 (m, 4H), 8.04 (d, 1H), 8.10 (bs, 1H); m/z: 728.3116.

### Example 4

### 1-(4-Fluorophenyl)-3-(R)-[3-(4-fluorophenyl)-3-hydroxypropyl]-4-(S)-[4-(N-{1-(R)-[N-(1-(S)-carboxy-2-hydroxyethyl)carbamoyl]-2-phenylethyl}carbamoylmethoxy)phenyl]azetidin-2-one

To the crude methanol mixture of 1-(4-fluorophenyl)-3-(R)-[2-(4-fluorobenzoyl)ethyl]-4-(S)-[4-(N-{1-(R)-[N-(1-(S)-carboxy-2-hydroxyethyl)carbamoyl]-2-phenylethyl}carbamoylmethoxy)phenyl]azetidin-2-one obtained from Method 8, sodium borohydride (10 mg, 0.26 mmol) was added. The reaction mixture was stirred at ambient temperature overnight. Ammonium acetate (5-10 mg) was added and the solvent was removed under reduced pressure. The residue was dissolved in MeCN and water. The crude product was purified with preparative BPLC on a C8-column. A gradient from 20 to 50% MeCN in 0.1M ammonium acetate buffer was used as eluent. MeCN and water were added and the resulting solution was lyophilised to give an off-white solid (16 mg, 46.6%). NMR (400 MHz, DMSO): 8.23 (d, 1H), 7.77 (d, 1H), 7.00-7.36 (m, 15H), 6.73-6.80 (m, 2H), 5.28 (bs, 1H), 4.82-4.87 (m, 1H) 4.62-4.58 (m, 1H), 4.56-4.45 (m, 1H), 4.40 (s, 2H), 3.75-3.89 (m, 1H), 3.47-3.54 (dd, 1H), 2.97-3.09 (m, 3H), 2.76-2.84 (dd, 1H), 1.77-1.87 (m, 1H), 1.66-1.76 (m, 3H); m/z: 700 (M−H)⁻.

### Examples 5-64

The following compounds could be prepared by the procedure of Example 4, but wherein different protecting groups may be used:

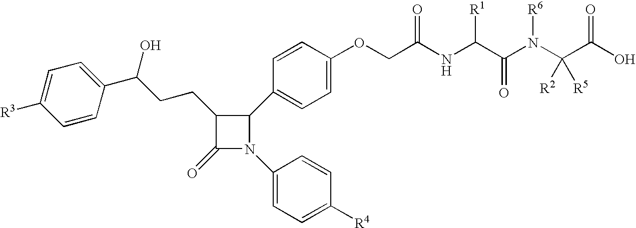

### Wherein R⁵ and R⁶ are hydrogen,

| Ex | R¹ | R² | R³ | R⁴ |
|---|---|---|---|---|
| 5 | H | Me | F | H |
| 6 | Ph | Me | F | H |
| 7 | H | Me | F | F |
| 8 | Ph | Me | F | F |
| 9 | H | Me | H | H |
| 10 | Ph | Me | H | H |
| 11 | H | —CH(Me)₂ | F | H |
| 12 | Ph | —CH(Me)₂ | F | H |
| 13 | Ph | —CH(Me)₂ | F | F |
| 14 | H | —CH(Me)₂ | H | H |
| 15 | Ph | —CH(Me)₂ | H | H |
| 16 | H | —CH₂CH(Me)₂ | F | H |
| 17 | Ph | —CH₂CH(Me)₂ | F | H |
| 18 | H | —CH₂CH(Me)₂ | F | F |
| 19 | Ph | —CH₂CH(Me)₂ | F | F |
| 20 | H | —CH₂CH(Me)₂ | H | H |
| 21 | Ph | —CH₂CH(Me)₂ | H | H |
| 22 | H | Ph | F | H |
| 23 | Ph | Ph | F | H |
| 24 | H | Ph | F | F |
| 25 | Ph | Ph | F | F |
| 26 | H | Ph | H | H |
| 27 | Ph | Ph | H | H |
| 28 | H | —CH₂Ph | F | H |
| 29 | Ph | —CH₂Ph | F | H |
| 30 | H | —CH₂Ph | F | F |
| 31 | Ph | —CH₂Ph | F | F |
| 32 | H | —CH₂Ph | H | H |
| 33 | Ph | —CH₂Ph | H | H |
| 34 | H | —CH₂(4-HOPh) | F | H |
| 35 | Ph | —CH₂(4-HOPh) | F | H |
| 36 | H | —CH₂(4-HOPh) | F | F |
| 37 | Ph | —CH₂(4-HOPh) | F | F |
| 38 | H | —CH₂(4-HOPh) | H | H |
| 39 | Ph | —CH₂(4-HOPh) | H | H |
| 40 | H | —CH₂OH | H | H |
| 41 | Ph | —CH₂OH | H | H |
| 42 | H | —CH₂COOH | F | F |
| 43 | H | —CH₂CH₂COOH | F | F |
| 44 | H | —CH₂CONH₂ | F | F |
| 45 | H | —CH₂CH₂CH₂CH₂NH₂ | F | F |
| 46 | H | —CH₂CH₂CH₂CH₂NHC(═NH)NH₂ | F | F |
| 47 | H | —CH(OH)Me (R) | F | F |
| 48 | H | 4-HOPh | F | F |
| 49 | H | —CH₂indol-3-yl | F | F |
| 50 | H | —CH₂-imidazol-4-yl | F | F |
| 51 | H | —CH₂CH₂SMe | F | F |
| 52 | H | cyclohexyl | F | F |
| 53 | Ph | —CH₂COOH | F | F |
| 54 | Ph | —CH₂CH₂COOH | F | F |
| 55 | Ph | —CH₂CONH₂ | F | F |
| 56 | Ph | —CH₂CH₂CH₂CH₂NH₂ | F | F |
| 57 | Ph | —CH₂CH₂CH₂CH₂NHC(═NH)NH₂ | F | F |
| 58 | Ph | —CH(OH)Me (R) | F | F |
| 59 | Ph | 4-HOPh | F | F |
| 60 | Ph | —CH₂indol-3-yl | F | F |
| 61 | Ph | —CH₂imidazol-4-yl | F | F |
| 62 | Ph | —CH₂CH₂SMe | F | F |
| 63 | Ph | cyclohexyl | F | F |
| 64 | Ph | —CH₂cyclohexyl | F | F |

### Example 65

### N-[(4-{(2S,3R)-1-(4-fluorophenyl)-3-[(3S)-3-(4-fluorophenyl)-3-hydroxypropyl]-4-oxoazetidin-2-yl}phenoxy)acetyl]glycyl-3-methyl-D-valine

(4-{(2S,3R)-1-(4-fluorophenyl)-3-[(3S)-3-(4-fluorophenyl)-3-hydroxypropyl]-4-oxoazetidin-2-yl}phenoxy)acetic acid (50 mg, 0.107 mmol) was dissolved in 5 ml DMF. N-Methylmorpholine (0.040 ml, 0.363 mmol) and TBTU (42 mg, 0.128 mmol) were added and the solution was stirred at 30° C. for 30 min. Glycyl-3-methyl-D-valine (neutral form of Method 2; 24 mg, 0.128 mmol) was added and the mixture was stirred over night. NH₄OAc was added. p-Xylene (3 ml) was added and the mixture was concentrated under reduced pressure. Toluene (ca 4 ml) was added and the mixture was concentrated until ca 1 ml remained. The mixture was purified using preparative HPLC on a C8 column (250×25 mm) using a gradient from 20% to 50% MeCN in 0.1M NE₄OAc buffer as eluent. Lyophilisation yielded the title compound in 10 mg (14%). The solid was dried in the vacuum oven at 40° C. for 2.5 h. M/z: 636 (M−1). ¹H-NMR (400 MHz, DMSO-d₆): 0.87 (s, 9H), 1.63-1.85 (m, 4H), 3.03-3.09 (m, 1H), 3.80 (d, 2H), 4.06 (d, 1H), 4.42-4.50 (m, 3H), 4.85 (d, 1H), 5.22 (brs, 1H), 6.94 (d, 2H), 7.01-7.13 (m, 4H), 7.14-7.20 (m, 2H), 7.23-7.33 (m, 4H), 7.86 (d, 1H), 8.22 (t, 1H).

### Example 66

### N-[(4-{(2S,3R)-1-(4-fluorophenyl)-3-[(3S)-3-(4-fluorophenyl)-3-hydroxypropyl]-4-oxoazetidin-2-yl}phenoxy)acetyl]glycyl-3-cyclohexyl-D-alanine

The title compound was prepared from glycyl-3-cyclohexyl-D-alanine according to the procedure described in example 65 and obtained in 19% yield. M/z: 676.5 (M−1). ¹H-NMR (400 MHz, DMSO-d₆): 0.68-1.86 (m, 17H), 3.03-3.09 (m, 1H), 3.73 (d, 2H), 4.08-4.16 (m, 1H), 4.4-4.50 (m, 3H), 4.85 (d, 1H), 5.25 (brs, 1H), 6.94 (d, 2H), 7.03-7.13 (m, 4H), 7.14-7.20 (m, 2H), 7.23-7.33 (m, 4H), 7.89-7.98 (m, 1H), 8.20 (t, 1H).

### Example 67

### N-[(4-{(2S,3R)-1-(4-fluorhenyl)-3-[3-(4-fluorophenyl)-3-hydroxypropyl]-4-oxozetidin-2-yl}phenoxy)acetyl]glycyl-D-valine

(4-{(2S,3R)-1-(4-fluorophenyl)-3-[3-(4-fluorophenyl)-3-oxopropyl]-4-oxoazetidin-2-ylphenoxy)acetic acid (33 mg, 0.071 mmol) was dissolved in 5 ml DCM and N-methylmorpholine (0.016 ml, 0.145 mmol) was added. TBTU (25 mg, 0.078 mmol) followed by tert-butyl glycyl-D-valinate hydrochloride (20 mg, 0.075 mmol) were added. The mixture was stirred for 1.5 days, extracted between DCM and brine and the organic phase was dried with Na₂SO₄ and concentrated. The intermediate tert-butyl ester (M/z: 678) was hydrolyzed using TFA (0.6 ml) in 3 ml DCM. The mixture was concentrated and dissolved in 3 ml MeOH. NaBH₄ (ca 20 mg) was added. Ammonium acetate buffer (0.1 M) was added and the mixture was purified using preparative HPLC on a C8 column. A stepwise gradient from 20-60% MeCN in 0.1 M ammonium acetate buffer was used as eluent. Lyophilisation yielded 33 mg (75%) of the title product. M/z: 606 (M-H₂O)⁺. ¹H-NMR (DMSO, 400 MHz): δ 0.75 (dd, 6H), 1.65-1.85 (m, 4H), 1.93-2.04 (m, 1H), 3.04-3.11 (m, 1H), 3.75 (d, 2H), 3.88 (dd, 1H), 4.45-4.55 (m, 4H), 4.85 (dd, 1H), 6.94-6.99 (m, 211), 7.05-7.14 (m, 4H), 7.16-7.22 (2H), 7.25-7.35 (m, 4H), 7.47 (d, 1H), 8.31 (t, 1H).

### Example 68

### N-[(4-{(2S,3R)-1-(4-fluorophenyl)-3-[(3S)-3-(4-fluorophenyl)-3-hydroxypropyl]-4-oxoazetidin-2-yl}phenoxy)acetyl]glycyl-D-valine

(4-{(2S,3R)-1-(4-fluorophenyl)-3-[(3S)-3-(4-fluorophenyl)-3-hydroxypropyl]-4-oxoazetidin-2-yl]phenoxy)acetic acid (63 mg, 0.135 mmol) was dissolved in 7 ml DCM. N-Methylmorpholine (0.045 ml, 0.408 mmol) and TBTU (50 mg, 0.155 mmol) were added and the solution was stirred for 10 min. Methyl glycyl-D-valinate hydrochloride (40 mg, 0.178 mmol) was added. After 30 min the mixture was extracted with brine containing some 0.3 M KHSO₄. The aqueous phase was extracted with 10 ml DCM and the combined organic phases were washed with brine, dried with Na₂SO₄ and concentrated to yield the intermediate methyl ester as a white solid. M/z: 636 (M−1). A mixture of 7.8 ml MeOH, 1.3 ml H₂O, 1.1 ml Et₃N and 0.060 ml 1,5-diazabicyclo-[4,3,0]-non-5-ene were added and the mixture was stirred for 1.5 h at ambient temperature and 1 h at 30° C. The solvent was partly evaporated under reduced pressure. The residue was purified by preparative HPLC using a C8 column and a gradient from 20-50% MeCN in 0.1 M ammonium acetate buffer as eluent. The title compound was obtained in 45 mg (53%). M/z: 622 (M−1). ¹H-NMR (DMSO, 400 MHz): δ 0.76 (dd, 6H), 1.64-1.85 (m, 4H), 1.93-2.04 (m, 1H), 3.04-3.11 (m, 1H), 3.76 (d, 2H), 3.87-3.95 (m, 1H), 4.45-4.53 (m, 4H), 4.86 (bs, 1H), 6.96 (d, 2H), 7.03-7.15 (m, 4H), 7.15-7.23 (2H), 7.24-7.36 (m, 4H), 7.51-7.61 (m, 1H), 8.30 (t, 1H).

Preparation of Starting Materials.

Method 1

1-(4-Fluorophenyl)-3-(R)-[2-(4-fluorobenzoyl)ethyl]-4-(S)-[4-(N-[(R)-1-carboxy-3-methylbutyl]carbamoylmethoxy)phenyl]azetidin-2-one

1-(4-Fluorophenyl)-3-(R)-[2-(4-fluorobenzoyl)ethyl]-4-(S)-[4-(carboxymethoxy)phenyl]azetidin-2-one (Method 6; 100 mg, 0.215 mmol), tert-butyl D-leucinate hydrochloride (53 mg, 0.237 mmol) and N-methylmorpholine (80 mg, 0.791 mmol) were dissolved in DCM (2 ml). TBTU (76 mg, 0.237 mmol) was added and the mixture was stirred for 60 minutes at room temperature. The solvent was evaporated and the residue was dissolved in formic acid (2 ml). The mixture was heated to 40-45° C. and kept over night at this temperature. The reaction mixture was evaporated under reduced pressure. The residue was purified by preparative HPLC using MeCN/ammonium acetate buffer (45:55) as eluent. After freeze-drying 115 mg (92%) of the title compound was obtained. The product was analyzed by LC-MS technique. M/z: 578.5.

Method 2

1-(4-Fluorophenyl)-3-(R)-[2-(4-fluorobenzoyl)ethyl]-4-(S)-{48 N-((R)-1-{N-1-(S)-(t-butoxycarbonyl)-2-(t-butoxy)ethyl]carbamoyl}-3-methylbutyl)carbamoylmethoxy]phenyl}azetidin-2-one

1-(4-Fluorophenyl)-3-(R)-[2-(4-fluorobenzoyl)ethyl]-4-(S)-[4-(N-[(R)-1-carboxy-3-methylbutyl]carbamoylmethoxy)phenyl]azetidin-2-one (Method 1; 40 mg, 0.069 mmol), tert-butyl O-(tert-butyl)-L-serinate hydrochloride (20 mg, 0.079 mmol) and N-methylmorpholine (25 mg, 0.25 mmol) were dissolved in DCM (1 ml). TBTU (26 mg, 0.081 mmol) was added and the mixture was stirred for 1 hour at room temperature. The reaction mixture was evaporated under reduced pressure and the residue was purified by column chromatography on silica gel. The product was eluted with DCMIEtOAc (50:50). 35 mg (65%) of the title compound was obtained. NMR (300 MHz): 0.93 (d, 6H), 1.13 (s, 9H), 1.45 (s, 9H), 1.5-1.7 (m, 4H), 2.2-2.5 (m, 2H), 3.1-3.35 (m, 3H), 3.52-3.56 (m, 1H), 3.73-3.77 (m, 1H), 4.49 (s, 2H), 4.51-4.70 (m, 4H), 6.67 (d, 1H), 6.90-7.30 (m, 11H), 7.96-8.01 (m, 2H).

Method 3

1-(4-Fluorophenyl)-3-(R)-[2-(4-fluorobenzoyl)ethyl]-4-(S)-{4-[N-((R)-1-{N-[1-(S)-(t-butoxycarbonyl)-3-(methyl)butyl]carbamoyl}-3-methylbutyl)carbamoylmethoxy]phenyl}azetidin-2-one

1-(4-Fluorophenyl)-3-(R)-[2-(4-fluorobenzoyl)ethyl]-4-(S)-[4-(N-[(R)-1-carboxy-3-methylbutyl]carbamoylmethoxy)phenyl]azetidin-2-one (Method 1; 25 mg, 0.043 mmol), tert-butyl L-leucinate hydrochloride (11 mg, 0.049 mmol) and N-methylmorpholine (20 mg, 0.20 mmol) were dissolved in DCM (1 ml). TBTU (17 mg, 0.053 mmol) was added and the mixture was stirred for 1 hour at room temperature. The solvent was evaporated under reduced pressure and the residue was purified by column chromatography on silica gel. The product was eluted with DCM/EtOAc (70:30). 30 mg.(93%) of the title compound was obtained. The product was analyzed by LC-MS technique. M/z: 747.8.

Method 4

1-(4-Fluorophenyl)-3-(R)-[2-(4-fluorobenzoyl)ethyl]-4-(S)-{4-[N-((R)-1-{N-[1-(R)-(t-butoxycarbonyl)-2-(phenyl)ethyl]carbamoyl}-3-methylbutyl)carbamoylmethoxy]phenyl}azetidin-2-one

1-(4-Fluorophenyl)-3-(R)-[2-(4-fluorobenzoyl)ethyl]-4-(S)-[4-(N-[(R)-1-carboxy-3-methylbutyl]carbamoylmethoxy)phenyl]azetidin-2-one (Method 1; 24 mg, 0.042 mmol), tert-butyl D-phenylalaninate hydrochloride (12 mg, 0.047 mmol) and N-methylmorpholine (15 mg, 0.15 mmol) were dissolved in DCM (0.5 ml). TBTU (15 mg, 0.047 mmol) was added and the mixture was stirred for 1 hour at room temperature. The solvent was evaporated under reduced pressure and the residue was purified by column chromatography on silica gel. The product was eluted with DCM(EtOAc (50:50). 15 mg (46%) of the title compound was obtained. The product was analyzed by LC-MS technique. M/z: 781.6.

Method 5

1-(4-Fluorophenyl)-3-(R)-[2-(4-fluorobenzoyl)ethyl]-4-(S)-[4-(t-butoxycarbonylmethoxy)phenyl]azetidin-2-one

A solution of 1-(4-fluorophenyl)-3-(R)-[2-(4-fluorobenzoyl)ethyl]4-(S)-(4-hydroxyphenyl)azetidin-2-one (Guangzhong Wu, YeeShing Wong, Xing Chen and Zhixian Ding, J. Org. Chem. 1999, 64, 3714; 1.00 g, 2.45 mmol), tert butyl bromoacetate (0.44 ml, 2.92 mmol) and Cs₂CO₃ (1.04 g, 3.19 mmol) in MeCN (5 ml) was stirred at RT for 4 hours. The solvent was removed under reduced pressure and the residue was partitioned between water (10 ml) and DCM (5 ml). The water layer was extracted once more with DCM (5 ml) and the combined organic layers where washed with brine, dried over MgSO₄ and concentrated. The residue was purified by flash chromatography on silica gel, using Hept:EtOAc (3:1) as eluent. This gave the desired product in 0.883 g (69%). NMR (DMSO-d₆, 500 MHz) 1.40 (s, 9H), 2.10-2.20 (m, 2H), 3.15-3.25 (m, 3H), 4.65 (s, 2H), 5.00 (d, 1H), 6.85-6.95 (m, 2H), 7.10-7.40 (m, 8H), 7.95-8.05 (m, 21); m/z: 522.2.

Method 6

1-(4-Fluorophenyl)-3-(R)-[2-(4-fluorobenzoyl)ethyl]-4-(S)-r4-(carboxmethoxy)phenyl]azetidin-2-one

A solution of 1-(4-fluorophenyl)-3-(R)-[2-(4-fluorobenzoyl)ethyl]-4-(S)-[4-(t-butoxycarbonylmethoxy)phenyl]azetidin-2-one (Method 5; 0.880 g, 1.69 mmol) in formic acid (5 ml) was stirred at room temperature for 20 hours. The solvent was removed under reduced pressure and the residue was dissolved in DCM (25 ml). The organic layer was washed twice with water (1×10 ml and 1×5 ml) and once with brine (5 ml), dried over MgSO₄ and concentrated. The desired product was obtained in 0.800 g (˜quantitative yield) as a white solid. NMR (DMSO-d₆, 500 MHz) 2.10-2.20 (m, 2H), 3.10-3.25 (m, 3H), 4.65 (s, 2H), 5.00 (d, 1H), 6.85-6.95 (m, 2H), 7.10-7.40 (m, 8H), 7.95-8.05 (m, 2H), 13.00 (bs, 1H); m/z: 466.2.

Method 7

1-(4-Fluorophenyl)-3-(R)-[2-(4-fluorobenzoyl)ethyl]-4-(S)-{4-[N-(1-(R)-carboxy-2-phenylethyl)carbamoylmethoxy]phenyl}azetidin-2-one

1-(4-Fluorophenyl)-3-(R)-[2-(4-fluorobenzoyl)ethyl]-4-(S)-[4-(carboxymethoxy)phenyl]azetidin-2-one (Method 6; 100 mg, 0.215 mmol) was dissolved in DCM (3 ml) followed by addition of (R)-phenylalanine-tert-butyl ester hydrochloride (62 mg, 0.258 mmol) and N-Methylmorpholine (71 μl, 0.644 mmol). TBTU (83 mg, 0.258 mmol) was added and the reaction mixture was stirred at ambient temperature overnight. The product mixture was extracted between DCM (5 ml) and water (3 ml), acidified to a pH of 2 using 2M KHSO₄. The organic phase was washed with water (2×3 ml), NaHCO₃ (5%) was added to a pH of 9 and it was washed once more with water (2×3 ml). The organic phase was dried over Na₂SO₄ and the solvent was evaporated to give a colourless oil (124 mg). The intermediate tert-butyl ester was confirmed. M/z 667 (M−H)⁻. Formic acid (2 ml) was added to the obtained crude oil and the mixture was stirred at ambient temperature overnight. The reaction mixture was heated at 50° C. for 2 hours and it was left at ambient temperature overnight. The solvent was removed under reduced pressure. MeCN was added and evaporated. The crude product was purified with preparative BPLC on a C8-column. A gradient from 20 to 50% MeCN in 0.1M ammonium acetate buffer was used as eluent. The collected fractions were concentrated under reduced pressure to remove the MeCN and lyophilised to give a white solid (80.3 mg, 61%). M/z: 613.

Method 8

1-(4-Fluorophenyl)-3-(R)-[2-(4-fluorobenzoyl)ethyl]-4-(S)-[4-(N-{1-(R)-[N-(1-(S)-carboxy-2-hydroxyethyl)carbamoyl]-2-phenylethyl}carbamoylmethoxy)phenyl]azetidin-2-one

1-(4-Fluorophenyl)-3-(R)-[2-(4-fluorobenzoyl)ethyl]-4-(S)-{4-[N-(1-(R)-carboxy-2-phenylethyl)carbamoylmethoxy]phenyl}azetidin-2-one (Method 7; 30 mg, 0.049 mmol) and tert-butyl O-(tert-butyl)-L-serinate hydrochloride (14.9 mg, 0.059 mmol) was added to DCM (1.5 ml). To this white suspension was added N-methylmorpholine (0.020 ml, 0.15 mmol) followed by TBTU (18.8 mg, 0.059 mmol). Additional TBTU (4 mg, 0.012 mmol) was added and the reaction mixture was stirred for 6 hours at ambient temperature. DCM (3 ml) and water (2 ml) were added and the mixture was acidified with 2M KHSO₄ to a pH of 2. The organic phase was washed with water (2×3 ml). NaHCO₃ (5%) was added to a pH of 9. The organic phase was washed with water (2×3 ml) and dried over Na₂SO₄. Filtration and removal of the solvent under reduced pressure afforded the crude ester. M/z: 812. Formic acid (1 ml) was added to the ester and the reaction mixture was stirred at ambient temperature overnight. The reaction was stirred for 7 hours at 40° C. and was then left to stand at ambient temperature for 48 hours. Analysis showed that the obtained product at this stage was the formate of the title compound, m/z: 728. The solvent was removed under reduced pressure and the residue was dissolved in toluene. The toluene was evaporated and the residue was dissolved in methanol (2 ml) and 6 drops of triethylamine was added. The reaction mixture was stirred at ambient temperature overnight yielding the title compound which was used without further purification. M/z: 700.

Method 9

(4-{(2S,3R)-1-(4-fluorophenyl)-3-[(3S)-3-(4-fluorophenyl)-3-hydroxypropyl]-4-oxoazetidin-2-yl}phenoxy)acetic acid

(3R,4S)-1-(4-fluorophenyl)-3-[(3S)-3-(4-fluorophenyl)-3-hydroxypropyl]-4-(4-hydroxyphenyl)azetidin-2-one (Guangzhong Wu, YeeShing Wong, Xing Chen and Zhixian Ding, J. Org. Chem. 1999, 64, 3714; 456 mg, 1.114 mmol) was dissolved in 10 ml MeCN. Cs₂CO₃ (400 mg, 1.228 mmol) was added followed by benzyl bromoacetate (0.21 ml, 1.283 mmol) at 0° C. The temperature was slowly raised to room temperature and the mixture was stirred over night, filtered and concentrated. The residue was extracted between EtOAc/diethyl ether and water. The organic phase was dried with Na₂SO₄, filtered and concentrated. The intermediate benzyl ester was obtained as an oil in 619 mg. ThF, 30 ml, and ca 10 wt % Pd/C were added. The mixture was stirred under H₂(g) at 1 atm pressure for 1.5 h. An additional spatula Pd/C was added and the mixture was hydrogenated for another 45 min. The mixture was filtered over celite. The filtrate was concentrated and the residue was purified using preparative HPLC on a C8 column (50×300 mm). A gradient from 20% to 40% MeCN in 0.1M ammonium acetate buffer was used as mobile phase. Lyophilization yielded 460 mg of an white solid (88% yield). ¹H-NMR (400 MHz, DMSO-d₆): 1.66-1.86 (m, 4H), 3.05-3.12 (m, 1H), 4.13 (s, 2H), 4.48 (t, 1H), 4.83 (d, 1H), 5.28 (brs, 1H), 6.79 (d, 2H), 7.08-7.16 (m, 4H), 7.18-7.33 (m, 6H).

Method 10

Glycyl-3-methyl-D-valine trifluoroacetate

To a 30° C. solution of N-(tezt-butoxycarbonyl)glycine (0.450 g, 2.569 mmol) and N-methylmorpholine (1.30 g, 12.84 mmol) in CH₂Cl₂ (50 ml) was added TBTU (0.99 g, 3.08 mmol). After 1.5 h, D-tert-leucine (0.303 g, 2.31 mmol) was added. After 30 minutes, the reaction was quenched by the addition of water (1 ml). The mixture was concentrated and the residue was purified through preparative HPLC using an eluent of 0-40% CH₃CN in 0.1M NH₄OAc buffer. Pure fractions were collected and concentrated. To the residue were added CH₂Cl₂ (10 ml) and TFA (3 ml). Full conversion to the corresponding aminoacid was obtained after 30 minutes. The reaction mixture was concentrated to give the desired compound (0.359 g, 46%) as a colourless solid. ¹H NMR (400 MHz, DMSO-d₆); 0.94 (s, 9H), 3.60-3.67 (m, 2H), 4.16 (d, 1H), 7.90-8.00 (m, 3H), 8.47 (d, 1H).

Method 11

Glycyl-3-cyclohexyl-D-alanine

N-(tert-butoxycarbonyl)glycine (2.0 g, 11.4 mmol) and DIPEA (4.0 g, 31 mmol) were dissolved in methylene chloride (25 ml). TBTU (4.1 g, 12.8 mmol) was added and the mixture was stirred for 15 min at room temperature. 3-cyclohexyl-D-alanine (2.1 g, 12.2 mmol) was added and the reaction mixture was stirred over night at room temperature. The reaction mixture was transferred to a separation funnel and was then extracted with a water/acetic acid solution (100 ml 5% acetic acid). The organic layer was separated and evaporated under reduced pressure. The residue was dissolved in formic acid (20 ml) and the mixture was stirred over night at 40° C. The formic acid was removed under reduced pressure. The residue was washed with water (50 ml) and then stirred in aceton (25 ml) for 1 h at room temperature. The solid material was filtered off and washed with aceton (20 ml). 530 mg (20%) of the title compound was obtained.

¹H-NMR (300 MHz, CD₃COOD): 0.8-1.9 (m, 13H), 3.94.1 (m, 2H), 4.55-4.65 (m, 1H).

Method 12

tert-butyl N-[(benzyloxy)carbonyl]glycyl-D-valinate

A mixture of N-[(benzyloxy)carbonyl]glycine (, 2.4g, 11.5 mmol), tert-butyl D-valinate hydrochloride (2.4 g, 11.4 mmol) and N-methylmorpholine (2.53 ml, 22.9 mmol) in DCM (20 ml) was stirred at room temperature. TBTU (4.79 g, 14.9 mmol) was added and the mixture was stirred for three days. The solvent was removed under reduced pressure. Water was added and the mixture was extracted two times with toluene. The organic layer was washed with brine, dryed (Na₂SO₄), filtered and concentrated. The crude product was purified by flash chromatography using DCM:EtOAc:aceton 4:1:1 as eluent to give 3.92 g (94%) of the title compound. NMR (500 MHz, CD₃COOD) 0.88-0.99 (m, 6H), 1.48 (s, 9H), 2.08-2.19 (m 1H), 3.85 (ABq, 2H), 4.24 (d, 1H), 5.12 (ABq, 2H), 7.28-7.41 (m, 5H).

Method 13

tert-butyl glycyl-D-valinate hydrochloride

tert-Butyl N-[(benzyloxy)carbonyl]glycyl-D-valinate (3.89 g, 10.7 mmol) and Pd on charcoal (5%, 0.3 g) were mixed in EtOH (95%, 80 ml) and stirred under H₂-atmosphere for 2 h. The mixture was filtered through Celite 521 and the solvent was evaporated under reduced pressure. MeCN (25 ml) and pyridine hydrochloride (1.25 g, 10.8 mmol) were added. The solvent was evaporated under reduced pressure to give 2.3g (81%) of the title product. NMR (500 MHz, CD₃COOD) 0.96-1.01 (m, 6H), 1.49 (s, 9H), 2.13-2.23 (m 1H), 3.76 (AB, 2H), 4.28-4.33 (m, 1H).

Method 14

Methyl glycyl-D-valinate hydrochloride

N-Benzylglycin (500 mg, 3.03 mmol) was dissolved in 20 ml DCM. DIPEA (1.55 ml, 9.08 mmol) and TBTU (1.17 g, 3.63 mmol) were added and the suspension was stirred for 10 min. D-Valine methyl ester hydrochloride (510 mg, 3.03 mmol) was added during 5 min and the mixture was stirred over night. Water (ca 10 ml) was added and the aqueous phase was acidified to pH 4 using 0.3 M KHSO₄. The yellow organic phase was washed with 10 ml acidified water (KHSO₄) followed by water, dried and concentrated. The crude mixture was purified by chromatography on 50 g SiO₂ using a gradient from 20-80% EtOAc in hexane as eluent. The intermediate (650 mg, 77%), was dissolved in MeOH and 25 mg Pd/C (10 mol %) was added. The solution was hydrogenated at 1 atm over night. The solution was filtered over celite and concentrated. MeCN, 10 ml, was added and the mixture was heated to ca 60° C. Pyridine hydrochloride (220 mg, 1.86 mmol) was added and the solution was slowly allowed to cool down. The solvent was removed under reduced pressure and the residue was recrystallized from 5 ml MeCN. The white solid was filtered off to yield the title compound in 360 mg (69%). ¹H-NMR (CD₃OD, 400 MHz): δ 0.92 (dd, 6H), 2.07-2.19 (m, 1H), 3.68 (s, 3H), 3.71 (s, 2H), 4.37 (d, 1H).

Examples of Intermediates of Formula (VI)

Method 15

1-(4-Fluorophenyl)-3-(R)-[3-(4-fluoroyhenyl)-3-hydroxypropyl]-4-(S)-[4-(N-[(R)-1-carboxy-3-methylbutyl]carbamoylmethoxy)phenyl]azetidin-2-one

1-(4-Fluorophenyl)-3-(R)-[2-(4-fluorobenzoyl)ethyl]4-(S)-[4-(N-[(R)-1-carboxy-3-methylbutyl]carbamoylmethoxy)phenyl]azetidin-2-one (Method 1; 25 mg, 0.043 mmol) was dissolved in methanol (0.5 ml). NaBH₄ (5 mg, 0.13 mmol) was added and the mixture was stirred for 15 minutes at room temperature. Two drops of acetic acid were added and the reaction mixture was evaporated under reduced pressure. The residue was purified by preparative HPLC using MeCN/ammonium acetate buffer (40:60) as eluent. After freeze-drying 16 mg (64%) of the title compound was obtained. The product was analyzed by LC-MS technique. M/z: 580.7.

Method 16

1-(4-Fluorophenyl)-3-(R)-[3-(4-fluorophenyl)-3-hydroxypropyl]-4-(S)-{4-[N-((R)-1-{N-[1-(S)-(t-butoxycarbonyl)-2-(t-butoxy)ethyl]carbamoyl}-3-methylbutyl)carbamoylmethoxy]phenyl}azetidin-2-one

1-(4-Fluorophenyl)-3-(R)-[2-(4-fluorobenzoyl)ethyl]-4-(S)-{4-[N-((R)-1-{N-[1-(S)-(t-butoxycarbonyl)-2-(t-butoxy)ethyl]carbamoyl}-3-methylbutyl)carbamoylmethoxy]phenyl}azetidin-2-one (Method 2; 10 mg, 0.0129 mmol) was dissolved in methanol (0.5 ml). NaBH₄ (3 mg, 0.079 mmol) was added and the mixture was stirred for 15 minutes at room temperature. The mixture was evaporated under reduced pressure and the residue was purified by column chromatography on silica gel. The product was eluted with DCM/EtOAc (50:50). 8 mg (80%) of the title compound was obtained. NMR (300 MHz): 0.93 (d, 6H), 1.13 (s, 9H), 1.45 (s, 9H), 1.8-2.0 (m, 4H), 2.2-2.35 (m, 1H), 3.05-3.15 (m, 1H), 3.52-3.56 (m, 1H), 3.73-3.77 (m, 1H), 4.49-4.75 (m, 6H), 6.67 (d, 1H), 6.90-7.32 (m, 13H).

Method 17

1-(4-Fluorophenyl)-3-(R)-[3-(4-fluoronhenyl)-3-hydroxypropyl]-4-(S)-4-[N-(1-(R)-carboxy-2-phenylethyl)carbamoylmethoxy]phenyl}azetidin-2-one

1-(4-Fluorophenyl)-3-(R)-[2-(4-fluorobenzoyl)ethyl]-4-(S)-{4-[N-(1-(R)-carboxy-2-phenylethyl)carbamoylmethoxy]phenyl}azetidin-2-one (Method 7; 19.8 mg, 0.032 mmol) was dissolved in methanol (1 ml) followed by addition of sodium borohydride (2.6 mg, 0.069 mmol). The reaction mixture was stirred at ambient temperature for 3 hours. Additional NaB (1 mg, 0.026 mmol) was added. Ammonium acetate (3 mg) was added and the solvent was removed under reduced pressure. The residue was purified by flash chromatography on silica gel (0.5 g) using 15% MeOH in DCM as eluent. The solvent was evaporated and the residue was dissolved in MeCN:water 1:1. The MeCN was removed under reduced pressure and the remaining solution was lyophilised to give a white solid (10 mg, 50 %). M/z: 613 (M−H)⁻.

1. A compound of formula (XV): 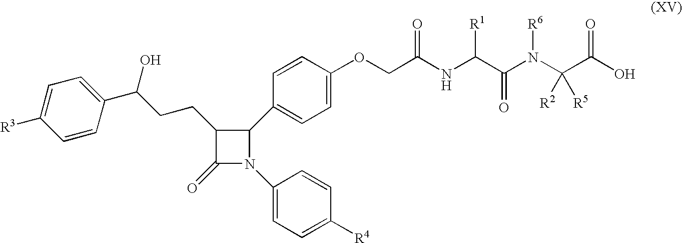
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆alkyl)₂amino, C₁-C₆ alkylcarbonylamino, C₁₋₆alkylS(O)ₐ wherein a is 0-2, C₃₋₆ cycloalkyl or aryl; and wherein any aryl group may be optionally substituted by one or two substituents selected from halo, hydroxy, C₁₋₆alkyl or C₁₋₆alkoxy;
R² and R⁵ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄)₃Si, N—(C₁₋₆alkyl)amino, N,N—(C₁₋₆alkyl)₂amino, C₁₋₆alkylS(O)ₐ, C₃₋₆cycloalkyl, aryl or arylC₁₋₆alkylS(O)ₐ, wherein a is 0-2; and wherein any aryl group may be optionally substituted by one or two substituents selected from halo, hydroxy, C₁₋₆alkyl or C₁₋₆alkoxy;
R³ is hydrogen, alkyl, halo, C₁₋₆alkoxy or C₁₋₆ alkylS—;
R⁴ is hydrogen, C₁₋₆ alkyl, halo or C₁₋₆alkoxy; and
R⁶ is hydrogen, C₁₋₆ alkyl, or arylC₁₋₆ alkyl;
wherein R⁵ and R² may form a ring with 2-7 carbon atoms and wherein R⁶ and R² may form a ring with 3-6 carbon atoms;
or a pharmaceutically acceptable salt, solvate, solvate of such a salt or a prodrug thereof.

2. A compound of formula (I): 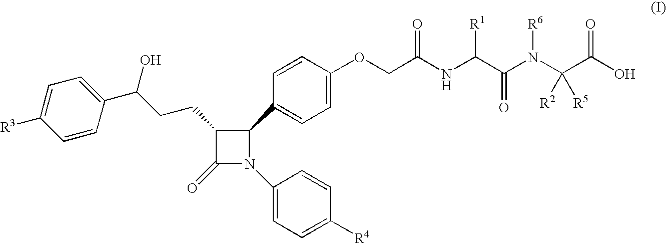
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆alkyl)₂amino, C₁-C₆ alkylcarbonylamino, C₁₋₆alkylS(O)ₐ wherein a is 0-2, C₃₋₆ cycloalkyl or aryl; and wherein any aryl group may be optionally substituted by one or two substituents selected from halo, hydroxy, C₁₋₆alkyl or C₁₋₆alkoxy;
R² and R⁵ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄)₃Si, N—(C₁₋₆alkyl)amino, N,N—(C₁₋₆alkyl)₂amino, C₁₋₆alkylS(O)ₐ, C₃₋₆cycloalkyl, aryl or aryl C₁₋₆alkylS(O)ₐ, wherein a is 0-2; and wherein any aryl group may be optionally substituted by one or two substituents selected from halo, hydroxy, C₁₋₆alkyl or C₁₋₆alkoxy;
R³ is hydrogen, alkyl, halo, C₁₋₆alkoxy or C₁₋₆ alkylS—;
R⁴ is hydrogen, C₁₋₆ alkyl, halo or C₁₋₆alkoxy; and
R⁶ is hydrogen, C₁₋₆ alkyl, or arylC₁₋₆ alkyl;
wherein R⁵ and R² may form a ring with 2-7 carbon atoms and wherein R⁶ and R² may form a ring with 3-6 carbon atoms;
or a pharmaceutically acceptable salt, solvate, solvate of such a salt or a prodrug thereof.

3. A compound according to claim 1, wherein:
R¹ is hydrogen, phenyl or a branched or unbranched C₁₋₆alkyl.

4. A compound according to claim 1, wherein:
R² is hydrogen, a branched or unbranched C₁₋₆alkyl, C₃₋₆cycloalkyl or aryl; wherein said C₁₋₆alkyl may be optionally substituted by one or more hydroxy, amino, acylamino, C₁₋₆alkoxyl, halo or methoxy C₁₋₆alkylS(O)ₐ wherein a is 0-2, C₃₋₆cycloalkyl or aryl; and wherein any aryl group may be optionally substituted by hydroxy, alkyl, alkoxy or cyano.

5. A compound according to claim 1, wherein:
R³ is hydrogen, halo, methyl or ethyl; wherein said methyl or ethyl may be optionally substituted by one or more C₁₋₆alkoxy, halo or methoxy.

6. A compound according to claim 1, wherein:
R³ is hydrogen, methyl, chlorine, fluorine, C₁₋₆ alkylS—, or methoxy.

7. A compound according to claim 1, wherein:
R⁴ is hydrogen or halo.

8. A compound according to claim 1, wherein:
R⁴ is chlorine or fluorine.

9. A compound according to claim 1, wherein:
R⁶ is hydrogen, C₁₋₆ alkyl, arylC₁₋₆alkyl or R⁶ and R² form a ring with 3-6 carbon atoms.

10. A compound according to claim 1, wherein:
R¹ is hydrogen;
R²is a branched or unbranched C₁₋₄alkyl, optionally substituted by a C₃₋₆cycloalkyl, alkylS—, aryl optionally substituted by hydroxy or cyano, amino, N—(C₁₋₆alkyl)amino, N,N—(C₁₋₆alkyl)₂amino or aryl C₁₋₆ alkylS(O)ₐ, wherein a is 0-2;
R³ and R⁴ are halo; and
R⁵ and R⁶ are hydrogen.

11. A compound of the formula (VI): 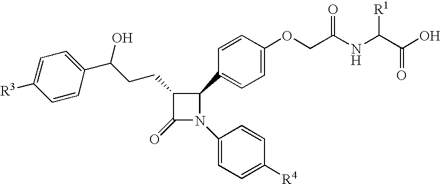
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl or C₁₋₆alkoxy;
R³ is hydrogen, alkyl, halo, C₁₋₆alkoxy or C₁₋₆ alkylS—; and
R⁴ is hydrogen, C₁₋₆ alkyl, halo or C₁₋₆alkoxy;
or a pharmaceutically acceptable salt, solvate, solvate of such a salt or a prodrug thereof.

12. A method of treating or preventing a hyperlipidemic condition comprising the administration of an effective amount of a compound according to claim 1 to a mammal in need thereof.

13. A method of treating or preventing atherosclerosis comprising the administration of an effective amount of a compound according to claim 1 to a mammal in need thereof.

14. A method for treating or preventing Alzheimers' disease comprising the administration of an effective amount of a compound according to claim 1 to a mammal in need thereof.

15. A method for treating or preventing a cholesterol associated tumor comprising the administration of an effective amount of a compound according to claim 1 to a mammal in need thereof.

16. A pharmaceutical formulation comprising a compound according to claim 1 in admixture with a pharmaceutically acceptable adjuvant, diluent and/or carrier.

17. A process for preparing a compound or a pharmaceutically acceptable salt, solvate, solvate of such a salt or a prodrug thereof comprising:
a) reacting a compound of formula (II): 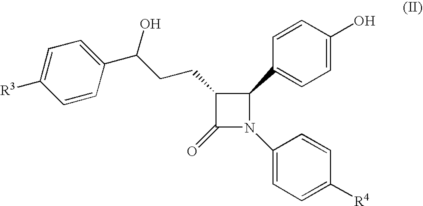
with a compound of formula (III): 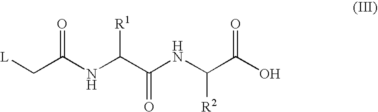
b) reacting an acid of formula (IV): 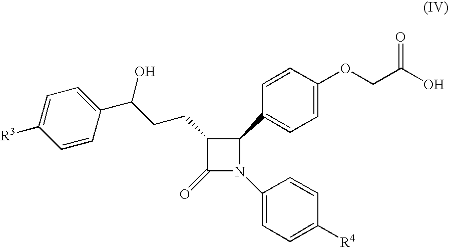
or an activated derivative thereof;
with an amine of formula (V): 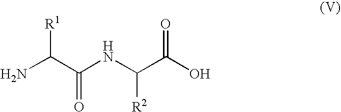
c) reacting an acid of formula (VI): 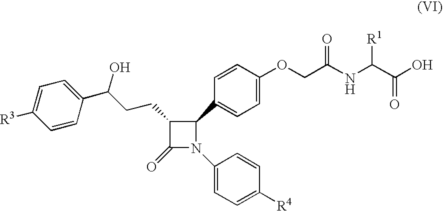
or an activated derivative thereof, with an amine of formula (VII): 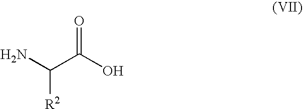
d) reducing a compound of formula (VIII): 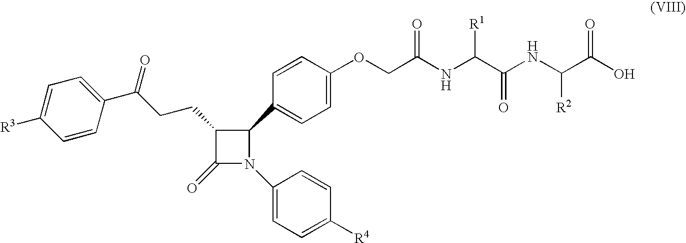
or e) De-esterifying a compound of formula (IX) 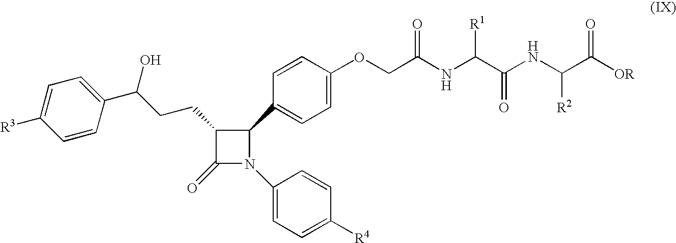
wherein the group C(O)OR is an ester group; an
wherein:
R¹ is hydrogen, C₁₋₆alkyl, C₃₋₆cycloalkyl or aryl: wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆alkyl))₂amino, C₁-C₆alkylcarbonylamino, optionally substituted by one or two substituents selected from halo, hydroxy, C₁₋₆alkyl or C₁₋₆alkoxy:
R² is hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄)_₃SI, N—(C₁₋₆alkyl)amino, N,N—(C₁₋₆alkyl)₂amino, C₁₋₆alkylS(O)ₐ, C₃₋₆cycloalkyl, aryl or aryl C₁₋₆alkylS(O)ₐ wherein a is 0-2; and wherein any aryl group may be optionally substituted by one or two substituents selected from halo, hydroxy, C₁₋₆alkyl or C₁₋₆alkoxy:
R³ is hydrogen, alkyl, halo, C₁₋₆alkoxy or C₁₋₆ alkylS—;
R⁴ is hydrogen, C₁₋₆alkyl halo or C₁₋₆alkoxy; and
L is a displaceable group;
and thereafter optionally:
i) converting a compound of the formula (I) into another compound of the formula (I);
ii) removing any protecting groups;
iii) forming a pharmaceutically acceptable salt, solvate, solvate of such a salt or a prodrug; or
iv) separating two or more enantiomers.

18. (canceled)

19. A combination of a compound according to formula (I) 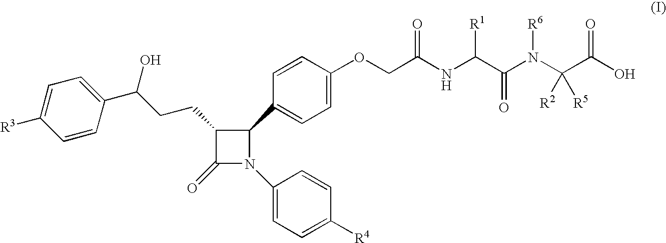
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆alkyl)₂amino, C₁₋₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl or C₁₋₆alkoxy:
R² and R⁵ are independently hydrogen, a branched or unbranched C₁₋₆alkyl C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁C₄)₃Si, N—(C₁₋₆alkyl)amino, N,N—(C₁₋₆alkyl)₂amino, C₁₋₆alkylS(O)ₐ, C₃₋₆cycloalkyl, aryl or aryl C₁₋₆alkylS(O)ₐ wherein a is 0-2; and wherein any aryl group may be optionally substituted by one or two substituents selected from halo, hydroxy, C₁₋₆alkyl or C₁₋₆alkoxy;
R³ is hydrogen, alkyl, halo, C₁₋₆alkoxy or C₁₋₆ alkylS—;
R⁴ is hydrogen, C₁₋₆alkyl, halo or C₁₋₆alkoxy; and
R⁶ is hydrogen, C₁₋₆alkyl or arylC₁₋₆ alkyl:
wherein R⁵ and R² may form a ring with 2-7 carbon atoms and wherein R⁶ and R² may form a ring with 3-6 carbon atoms;
or according to formula (XV) 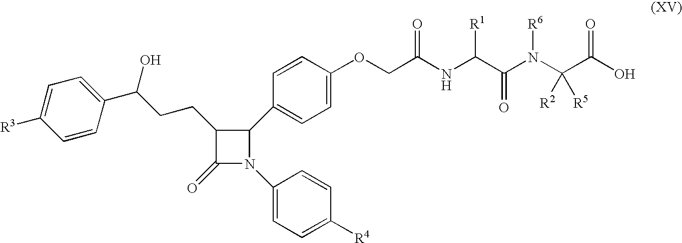
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆ alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl or C₁₋₆alkoxy;
R² and R⁵ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄)₃Si, N—(C₁₋₆alkyl)amino, N,N—(C₁₋₆alkyl)₂amino, C₁₋₆alkylS(O)ₐ, C₃₋₆cycloalkyl, aryl or arylC₁₋₆alkylS(O)ₐ wherein a is 0-2; and wherein any aryl group may be optionally substituted by one or two substituents selected from halo, hydroxy C₁₋₆alkyl or C₁₋₆alkoxy;
R³ is hydrogen, alkyl, halo, C₁₋₆alkoxy or C₁₋₆ alkylS—;
R⁴ is hydrogen, C₁₋₆alkyl halo or C₁₋₆alkoxy: and
R⁶ is hydrogen, C₁₋₆ alkyl or arylC₁₋₆alkyl;
wherein R⁵ and R² may form a ring with 2-7 carbon atoms and wherein R⁶ and R² may form a ring with 3-6 carbon atoms;
with a PPAR alpha and/or gamma agonist.

20. A combination of a compound according to formula (I) 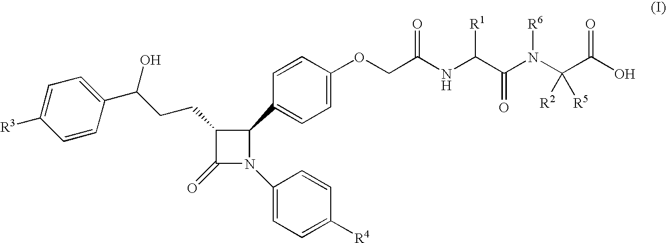
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆alkyl)₂amino, C₁-C₆alkylcarbonylamino, C₁₋₆alkylS(O)ₐ wherein a is 0-2, C₃₋₆cycloalkyl or aryl; and wherein any aryl group may be optionally substituted by one or two substituents selected from halo, hydroxy, C₁₋₆alkyl or C₁₋₆alkoxy:
R² and R⁵ are independently hydrogen, a branched or unbranched C₁₋₆alkyl, C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁-C₄)₃Si, N—(C₁₋₆alkyl)amino, N,N—(C₁₋₆alkyl)₂amino, C₁₋₆alkylS(O)ₐ, C₃₋₆cycloalkyl, aryl or aryl C₁₋₆alkylS(O)ₐ wherein a is 0-2; and wherein any aryl group may be optionally substituted by one or two substituents selected from halo, hydroxy, C₁₋₆alkyl or C₁₋₆alkoxy;
R³ is hydrogen, alkyl, halo, C₁₋₆alkoxy or C₁₋₆ alkylS—;
R⁴ is hydrogen, C₁₋₆alkyl halo or C₁₋₆alkoxy: and
R⁶ is hydrogen, C₁₋₆ alkyl or arylC₁₋₆alkyl;
wherein R⁵ and R² may form a ring with 2-7 carbon atoms and wherein R⁶ and R² may form a ring with 3-6 carbon atoms;
or according to formula (XV) 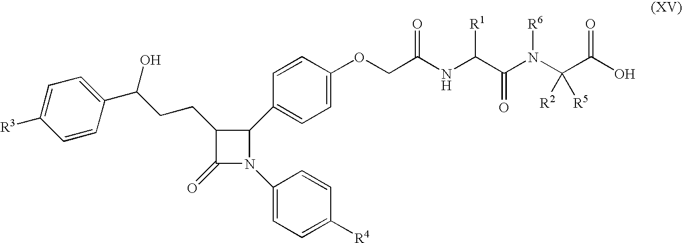
wherein:
R¹ is hydrogen, C₁₋₆alkyl, C₃₋₆cycloalkyl or aryl; wherein said C₁₋₆alkyl may be optionally substituted by one or more hydroxy, amino, guanidino, carbamoyl, carboxy, C₁₋₆alkoxy, N—(C₁₋₆alkyl)amino, N,N—(C₁₋₆alkyl)₂amino, C₁₋₆alkylcarbonylamino, C₁₋₆alkylS(O)ₐ, wherein a is 0-2, C₃₋₆cycloalkyl or aryl; and wherein any aryl group may be optionally substituted by one or two substituents selected from halo, hydroxy, C₁₋₆alkyl or C₁₋₆alkoxy:
R² and R⁵ are independently hydrogen, a branched or unbranched C₁₋₆alkyl C₃₋₆cycloalkyl or aryl; wherein said C₁₋₆alkyl may be optionally substituted by one or more hydroxy, amino, guanidino, cyano, carbamoyl, carboxy, C₁₋₆alkoxy, aryl C₁₋₆alkoxy, (C₁C₄)₃Si, N—(C₁₋₆alkyl)amino, N,N—(C₁₋₆alkyl)₂amino, C₁₋₆alkylS(O)ₐ, C₃₋₆cycloalkyl, aryl or arylC₁₋₆alkylS(O)ₐ wherein a is 0-2; and wherein any aryl group may be optionally substituted by one or two substituents selected from halo, hydroxy C₁₋₆alkyl or C₁₋₆alkoxy;
R³ is hydrogen, alkyl, halo, C₁₋₆alkoxy or C₁₆ alkylS—;
R⁴ is hydrogen, C₁₋₆alkyl halo or C₁₋₆alkoxy: and
R⁶ is hydrogen, C₁₋₆alkyl or arylC₁₋₆alkyl:
wherein R⁵ and R² may form a ring with 2-7 carbon atoms and wherein R⁶ and R² may form a ring with 3-6 carbon atoms;
with an HMG Co-A reductase inhibitor.

21. A method of treating or preventing a hyperlipidemic condition comprising the administration of an effective amount of a compound according to claim 11 to a mammal in need thereof.

22. A method of treating or preventing atherosclerosis comprising the administration of an effective amount of a compound according to claim 11 to a mammal in need thereof.

23. A method for treating or preventing Alzheimers' disease comprising the administration of an effective amount of a compound according to claim 11 to a mammal in need thereof.

24. A method for treating or preventing a cholesterol associated tumor comprising the administration of an effective amount of a compound according to claim 11 to a mammal in need thereof.

25. A pharmaceutical formulation comprising a compound according to claim 11 in admixture with a pharmaceutically acceptable adjuvant, diluent and/or carrier.

26. A process according to claim 17 wherein L is a halogen or sulphonyloxy group.

27. A process according to claim 26 wherein L is a chloro, bromo, methanesulfonyloxy or toluene-4-sulphonyloxy group.

28. A process according to claim 17 wherein the C(O)OR ester group is methoxycarbonyl, ethoxycarbonyl, t-butoxycarbonyl, or benzyloxycarbonyl.
